(19) United States
(12) Patent Application Publication
(10) Pub. No.: US 2024/0182854 A1
(43) Pub. Date: Jun. 6, 2024

(54) Methods to Expand a T Regulatory Cell Master Cell Bank

(71) Applicants: The Trustees of the University of Pennsylvania, Philadelphia, PA (US); Regents of the University of Minnesota, St. Paul, MN (US)

(72) Inventors: James L. Riley, Downingtown, PA (US); Carl H. June, Merion Station, PA (US); Bruce R. Blazar, Golden Valley, MN (US); Keli Hippen, Robbinsdale, MN (US)

(21) Appl. No.: 18/450,234

(22) Filed: Aug. 15, 2023

Related U.S. Application Data
(63) Continuation of application No. 16/366,004, filed on Mar. 27, 2019, now abandoned.
Application No. 16/366,004 is a continuation of application No. 13/639,927, filed on Jan. 9, 2013, now abandoned.
(60) Provisional application No. 61/322,186, filed on Apr. 8, 2010.

(51) Int. Cl. C12N 5/0783

(52) U.S. Cl. CPC C12N 5/0637; C12N 2500/32; C12N 2501/04; C12N 2501/2302; C12N 2501/51; C12N 2501/515

(57) ABSTRACT

The present invention provides compositions and methods for expanding natural T regulatory cells (nTregs) without substantially sacrificing suppressive function of the cells. Accordingly, the invention provides uses of the expanded nTregs for cellular therapy.

### CROSS-REFERENCE TO RELATED APPLICATIONS

The present application is a continuation of U.S. patent application Ser. No. 16/366,004, filed Mar. 27, 2019, abandoned, which is a continuation of U.S. patent application Ser. No. 13/639,927, filed Jan. 9, 2013, abandoned, which is a 35 U.S.C. § 371 national phase application from, and claims priority to, International Application No. PCT/US2011/030194, filed Mar. 28, 2011, which claims priority under 35 U.S.C. § 119(e) to U.S. Provisional Application No. 61/322,186, filed Apr. 8, 2010, all of which applications are incorporated herein by reference in their entireties.

### STATEMENT REGARDING FEDERALLY SPONSORED RESEARCH OR DEVELOPMENT

This invention was made with government support under grant numbers CA105216 and CA067493 awarded by the National Institutes of Health. The government has certain rights in the invention.

### BACKGROUND OF THE INVENTION

Acute graft-versus-host disease (GVHD) is a significant cause of morbidity and mortality after hematopoietic cell transplantation (Welniak et al., 2007, Annu Rev Immunol 25:139-70). Natural regulatory T-cells (nTregs) express the transcription factor FoxP3, and are required for immune self-tolerance (Wildin et al., 2005, Journal of Autoimmunity 25 Suppl:56-62). In murine models, adoptive transfer of nTregs prevents GVHD, donor bone marrow graft rejection, and speeds immune recovery in GVHD-prone animals (Hoffmann et al., 2002, J Exp Med 196(3):389-99; Shevach et al., 2006, Immunol Rev 212:60-73; Taylor et al., 2002, Blood 99:3493-9), making Tregs an attractive therapeutic tool for preventing and/or treating disease in humans (Gavin, et al., 2003, Current Opinion in Immunology 15:690-6; June et al., 2006, Semin Immunol 18:78-88; Piccirillo et al., 2004, Seminars in Immunology 16:81-8; Roncarolo et al., 2007, Nat Rev Immunol 7:585-98). Clinical testing has been hampered by low nTreg frequency (1-2%) in peripheral blood (PB) (Baecher-Allan et al., 2001, J Immunol 167:1245-53), contamination with non-Tregs, CD25 T-effector or -memory cells (June et al., 2006, Semin Immunol 18:78-88; Godfrey et al., 2004, Blood 104(2):453-61), and availability of good manufacturing practice (GMP)-compatible procedures for nTreg purification. Maximizing yield is also critical, as murine studies find high Treg doses (˜1:1 with donor T-cells) are required to efficiently and reproducibly suppress GVHD (Taylor et al., 2002, Blood 99:3493-9).

Peripheral blood contains a small population of T cell lymphocytes that express the T regulatory phenotype (“Treg”), i.e., positive for both CD4 and CD25 antigens. There are several subsets of Treg cells (Bluestone et al., 2003 Nature Rev. Immunol. 3: 253). One subset of regulatory cells develops in the thymus. Thymic derived Treg cells function by a cytokine-independent mechanism, which involves cell to cell contact (Shevach, 2002 Nature Rev. Immunol 2: 389). They are essential for the induction and maintenance of self-tolerance and for the prevention of autoimmunity (Shevach, 2000 Annu. Rev. Immunol. 18: 423-449). These regulatory cells prevent the activation and proliferation of autoreactive T cells that have escaped thymic deletion or recognize extrathymic antigens, thus they are critical for homeostasis and immune regulation, as well as for protecting the host against the development of autoimmunity. Thus, immune regulatory CD4 CD25 T cells are often referred to as “professional suppressor cells.”

Naturally arising CD4 CD25 Treg cells are a distinct cell population of cells that are positively selected on high affinity ligands in the thymus and that have been shown to play an important role in the establishment and maintenance of immunological tolerance to self antigens. Deficiencies in the development and/or function of these cells have been associated with severe autoimmunity in humans and various animal models of congenital or induced autoimmunity.

Treg cells manifest their tolerogenic effects directly via cell-to-cell contact or indirectly via soluble factors. Although the suppressive mechanisms of these cells remain to be fully elucidated, blockade of IL-2 expression in effector T cells (Teff), physical elimination of Teff cells, induction of tolerogenic dendritic cells (DCs) via CTLA-4/B7 axis, and inhibition of Teff cells via TGF-β and IL-10 are some of the mechanisms that have been implicated to date. It also has been shown that reverse signaling through CTLA-4/CD80 into Teff cells plays an important role in their inhibition by Treg cells. Similarly, interactions between CTLA-4 on Treg cells and CD80 on DCs can result in reverse signaling and upregulation of the indoleamine dioxygenase enzyme that is involved in tolerance via the regulation of tryptophan metabolism.

Treg cells can also be generated by the activation of mature, peripheral CD4 T cells. Studies have indicated that peripherally derived Treg cells mediate their inhibitory activities by producing immunosuppressive cytokines, such as transforming growth factor-beta (TGF-β) and IL-10 (Kingsley et al., 2002 J. Immunol. 168: 1080; Nakamura et al., 2001 J. Exp. Med. 194: 629-644). Treg are have been described in the literature as being hypoproliferative in vitro (Sakaguchi, 2004 Ann. Rev. Immunol. 22: 531). Trenado et al. provided the first evaluation of the therapeutic efficacy of ex vivo activated and expanded CD4 CD25 regulatory cells in an in vivo mouse model of disease (Trenado et al., 2002 J. Clin. Invest. 112(11): 1688-1696).

Natural Tregs were found to be more readily purified from umbilical cord blood (UCB) than peripheral blood (PB) due to the relative paucity of CD25⁺ non-Tregs in UCB, and could be expanded several hundred fold ex vivo using anti-CD3/CD28 mAb-coated microbeads and IL-2 (Godfrey et al., 2004, Blood 104(2):453-61; Hippen et al., 2008, Blood 112(7):2847-57). These studies facilitated the world's first clinical trial to study the safety of ex-vivo expanded nTregs. No adverse effects were observed and a trend towards a lower incidence of acute grade II-IV GVHD was observed, but, the maximum cell dose was limited by insufficient and variable nTreg expansion rates for some UCB units (Brunstein et al., 2011, Blood 117(3): 1061-70). It has been shown that stimulation of UCB nTregs with cell-based aAPC increases expansion (˜4-fold) over bead-based aAPC, although this degree of expansion was less than desired. Because the number of nTregs in UCB are limited and the dose-limiting toxicity was not reached, other sources of nTregs need to be explored to determine the maximal efficacy of single or multiple dose Treg therapy.

However, the inadequacy of isolation and expansion methods used for the generation of Treg cell lines has significantly interfered with advances in the research on human Treg cells. Thus, there is a need for methods of producing sufficient number of these Treg cells to permit characterization and to provide for safe and effective therapeutic use in human patients. There also remains a need for large-scale expansion of human Treg cells for clinical trials including, but not limited to immunotherapy or immunosuppression of cancers, particularly solid tumor cancers. Equally important is a need to suppress in vivo alloresponses and autoimmune responses, such as, although not limited to, graft-vs-host disease (GVHD).

### SUMMARY OF THE INVENTION

The invention provides a method of expanding a population of cells comprising natural T regulatory cells (nTregs). In one embodiment, the method comprises 1) culturing the population of cells comprising nTregs in a culture medium comprising a first agent that provides a primary activation signal to T cells and a second agent that provides a co-stimulatory signal to T cells; 2) monitoring proliferation of the nTregs; and 3) re-stimulating the nTregs when the rate of nTreg proliferation has decreased based upon a desired cell size, thereby inducing further proliferation the nTregs.

In one embodiment, the population of cells is cultured in the presence of Rapamycin.

In another embodiment, the population of cells is re-stimulated in the presence of Rapamycin.

In another embodiment, the population of cells is cultured and re-stimulated in the presence of Rapamycin.

In one embodiment, the desired cell size is the same size as a resting nTreg.

In one embodiment, the first agent is anti-CD3 antibody.

In another embodiment, the second agent is a molecule that binds CD28. In one embodiment, the molecule that binds CD28 is selected from the group consisting of anti-CD28 antibody, B7 (CD80), B7-2 (CD86), and any combination thereof.

In one embodiment, the method further comprises repeating steps 1 through 3 at least once to produce a population of nTreg cells that is increased in cell number from about 100- to about 10,000,000-fold compared with the original nTreg cell population.

In one embodiment, the expanded population of cells substantially retains a nTreg phenotype. In another embodiment, the expanded population of cells exhibits FoxP3 profile indicative of a nTreg and exhibits suppressor activity. In yet another embodiment, the expanded population of cells does not secrete IFNγ and IL-2. In one embodiment, the expanded population of cells has not substantially reverted to T effector phenotype.

In one embodiment, the population of cells has been isolated from an umbilical cord blood sample prior to culturing in the medium.

In another embodiment, the population of cells has been isolated from a peripheral blood sample prior to culturing in the medium.

In one embodiment, the population of cells has been isolated by flow-sorting prior to culturing in the medium.

In one embodiment, the population of cells has been cryopreserved.

The invention provides an isolated population of cells expanded according to the methods of the invention.

The invention also provides a method for inhibiting proliferation of a T cell comprising contacting the T cell with a nTreg expanded according to the methods of the invention.

The invention also provides a kit for expanding a population of cells comprising nTregs. In one embodiment, the kit comprises an antibody that specifically binds CD3 bound to a physical support, a molecule that binds CD28 bound to a physical support, an applicator, and an instructional material for the use thereof.

The invention also provides a method for adoptive transfer therapy. In one embodiment, the method comprises administering an expanded population of cells comprising nTregs to a mammal in need thereof to prevent or treat an immune reaction that is adverse to the mammal, wherein the expanded population of cells has been expanded according to a method comprising 1) culturing a population of cells comprising nTregs in a culture medium comprising a first agent that provides a primary activation signal to T cells and a second agent that provides a co-stimulatory signal to T cells; 2) monitoring proliferation of the nTregs; and 3) re-stimulating the nTregs when the rate of nTreg proliferation has decreased based upon a desired cell size, thereby inducing further proliferation the nTregs.

The invention provides a method of treating a disease or condition associated with enhanced immunity. In one embodiment, the method comprises administering an expanded population of cells comprising nTregs to a mammal in need thereof, wherein the expanded population of cells has been expanded according to a method comprising 1) culturing a population of cells comprising nTregs in a culture medium comprising a first agent that provides a primary activation signal to T cells and a second agent that provides a co-stimulatory signal to T cells; 2) monitoring proliferation of the nTregs; and 3) re-stimulating the nTregs when the rate of nTreg proliferation has decreased based upon a desired cell size, thereby inducing further proliferation the nTregs.

In one embodiment, the disease or condition associated with enhanced immunity is selected from the group consisting of an autoimmune disease, graft versus host disease, and graft rejection.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic of a representative expansion scheme for culturing Tregs in the presence of Rapamycin and restimulation of the Tregs based upon cell size.

FIG. 2 is a series of images demonstrating cell size guidance of expansion of Tregs using anti-CD3/28 beads versus KT64/86 cells.

FIG. 3 is a series of images demonstrating that cell size guided restimulation dramatically increases fold expansion of Tregs.

FIG. 4 is a series of images demonstrating retention of Treg phenotype despite dramatic expansion.

FIG. 5 is a schematic demonstrating Milentyi bead enriched and cell sorted isolated Tregs.

FIG. 6 is an image depicting dramatic expansion of highly purified Tregs in the presence of Rapamycin.

FIG. 7 is a series of images depicting fold expansion after each round of restimulation.

FIG. 8 is a series of images demonstrating that Treg phenotype and suppression is retained despite dramatic expansion of highly purified Tregs.

FIG. 9 is a series of images demonstrating that expanded Tregs still suppress anti-CD3 mAb induced CD8 proliferation.

FIG. 10 is a schematic of a representative expansion scheme for culturing Tregs in the absence of Rapamycin and restimulation of the Tregs based upon cell size.

FIG. 11 is a series of images demonstrating that the loss of FoxP3 and suppressor function of Tregs in the absence of Rapamycin.

FIG. 12 is a series of images demonstrating that Tregs expanded in the presence of Rapamycin have low levels of proinflammatory cytokines after restimulation.

FIG. 13 is an image depicting umbilical cord blood (UCB)-Treg production with restimulation.

FIG. 14 is a schematic of a timeline for an in vivo XGVHD experiment using re-stimulated UCB nTreg.

FIG. 15 is a series of images depicting intracellular cytokine staining for sort-purified, restimulated nTreg.

FIG. 16 is a series of images demonstrating that re-stimulation greatly increases PB nTreg expansion, and cell-based aAPC are more effective than bead-based aAPC in expanding PB nTreg. Regulatory T cells were purified from peripheral blood Leukapheresis products and expanded using GMP anti-CD3/28 beads or CD3-loaded cell line (KT64/86). (A) depicts a representative GMP purification scheme. (B) depicts a scheme showing time course of experiment and ranges for size-based re-stimulation (R0 =no re-stimulation R1=one re-stimulation, etc.). Fold nTreg expansion (average±SEM); Total (C) or following each round of stimulation (D). (E) depicts the percent of cultured cells (CD4-gated) that are CD127⁻Foxp3⁺ after each round of stimulation. (F) depicts percent suppression of in vitro, anti-CD3 mediated CD8 T cell proliferation at 1:4 (nTreg:PBMNC) as determined via CFSE dye dilution. (G) depicts nTreg re-stimulated with PMA and Ionomycin for 4 hours in the presence of Brefeldin A, and the percent of cells secreting IL-2 or IFNγ was determined by flow cytometry. (H) depicts bead-purified PB nTreg re-stimulated 3 or 4 times (black and gray symbols, respectively) with anti-CD3 loaded KT 64/86 harvested and genomic DNA purified and Foxp3 TSDR demethylation status assessed using bisulfite sequencing, compared to nTreg purity (% of CD4+ that are CD127-Foxp3) or % suppression at 1:4 (nTreg:PBMNC). Averages are for three independent experiments, individual symbols in (E) and (F) represent independent experiments. Individual brackets indicate the range of days for each stimulus. * represents p<0.05.

FIG. 17 is a series of images demonstrating that increased initial purity decreases loss of suppressive function when nTreg are expanded with repetitive stimulations in the presence of Rapamycin. PB nTreg were sort-purified (CD4⁺25⁺⁺127⁻) and expanded with CD3-loaded KT64/86 in the presence or absence of Rapamycin using 4 or 2 re-stimulations, respectively. (A) depicts a representative scheme showing time course of the experiment and time ranges for size-based re-stimulation (R0=no re-stimulation R1=one re-stimulation, etc.); individual brackets indicate the range of days for each stimulus. (B) depicts average cell size (±SEM) over time for PB nTreg cultures re-stimulated±Rapamycin. Representative examples (C) and average (D) expansion of nTreg±Rapamycin, respectively, are depicted. Arrows in (C) on days 25 and 55 mark two distinct phases (plateau and growth phase) seen after first re-stimulation of nTreg cultures grown without Rapamycin. Average % CD127−Foxp3+(CD4-gated) or % suppression of in vitro T cell proliferation at 1:4 (Treg:PBMNC) for cultures expanded in the absence (E) and (F) or presence (G) and (H) of Rapamycin, respectively. Bars represent average, other symbols represent individual experiments.

FIG. 18 is a series of images depicting cytokine production by PB nTreg re-stimulated with or without Rapamycin. PB nTreg were sort-purified and expanded with multiple rounds of stimulation with anti-CD3 loaded KT 64/86 in the presence or absence of Rapamycin. The R1 without Rapamycin sample corresponds to the day 25 time point with high Foxp3 staining. (A) depicts a representative example of cytokine production by Foxp3+ and − cells (CD4-gated). Average (±SEM) % of cells secreting IL-2 (B), IFNγ (C), IL-4 (D), or IL-17 (E) are depicted. Averages are for three independent experiments.

FIG. 19 is a series of images demonstrating that PB nTreg expanded over 50 million fold can still ameliorate disease in a xenogeneic model of GVHD, even after freezing and thawing. (A) depicts the summary of purity (% of CD4+ cells that are CD127−Foxp3+), and in vitro suppressive function for in vitro expanded nTreg or CD4+25− (grown±TGFβ) with a single stimulation with KT64/86 cells. (B) depicts a Kaplan-Meyer survival curve comparing NOD/Scid/gc^{-/-} mice receiving human PBMNC only or co-transferred with nTreg, CD4+25− cells, or CD4+25− cells expanded in TGFβ co-transferred at 1:1 (e.g. 30×10⁶ PBMNC and 30×10⁶ nTreg). (C) depicts the summary of fold expansion, purity (% of CD4+ cells that are CD127−Foxp3+), and in vitro suppressive function for nTreg expanded with 4 re-stimulations (R4). (D) depicts a Kaplan-Meyer survival curve comparing NOD/Scid/gc^{-/-} mice receiving human PBMNC ±fresh nTreg re-stimulated 4 times (R4) co-transferred at 1:1 (30×10⁶ PBMNC and 30×10⁶ nTreg). FIG. 19E depicts the average weight (% of initial) for mice surviving on a given day for different groups of mice. (*) p≤0.05 for fresh nTreg from days 14-21. (F) depicts the summary of fold expansion, purity (% of CD4+ cells that are CD127-Foxp3+), and in vitro suppressive function for expanded nTreg re-stimulated 3 or 4 times (R3 and R4, respectively). (G) depicts a Kaplan-Meyer survival curve showing survival of mice receiving human PBMNC±cryopreserved and thawed R3 or R4 nTregs (HLA-A2) co-transferred at 1:2 (i.e. 15×10⁶ nTreg and 30×10⁶ PBMNC). n=10, 8 and 7 for groups PBMNC, R3 nTreg and R4 nTreg, respectively. (H) depicts the average number (±SEM) of human CD4 HLA-A2, CD8 HLA-A2 or Total CD4/8A2-cells per μl blood on day 30 for animals in (G)).

FIG. 20 is a series of images demonstrating that nTreg stimulated with cell-based aAPC have increased peak size, but decreased Foxp3. PB nTreg were bead purified and expanded with multiple rounds of stimulation with CD3/28 beads or a CD3-loaded cell line (KT64/86). (A) depicts a representative example of FSC vs. SSC, CD4 vs. CD25, and CD127 versus Foxp3 (CD4-gated) profiles before and after bead-based purification. (B) depicts the viability of nTreg cultures expanded after a single stimulation with anti-CD3/28 beads or KT64/86 cells. Granzyme B production in nTreg stimulated with anti-CD3/28 beads or KT64/86 cells as assessed by intracellular cytokine staining before or after re-stimulation with PMA/Ionomycin (C) or in culture supernatant assessed by ELISA (D). On culture days, cell size was determined and is represented as averages (±SEM) over time (E) or as peak cell size (F). Representative Foxp3 staining (G) and relative CD25 staining (average±SEM) (H) for Foxp3+ cells expanded with either CD3/28 beads or KT64/86 (i.e. CD3/28 MFI/KIT64/86 MFI) is depicted. (I) demonstrates that IL-17 production by PB nTreg re-stimulated with CD3/28 beads or KT64/86 was assessed by intracellular staining following a 4 hour treatment with PMA/Ionomycin. Cells were also stained for Foxp3, and the average (±SEM) % IL-17+ cells of each type shown (n=3). (J) demonstrates that the relative in vivo suppressive function of nTreg expanded with multiple stimulations using CD3/28 beads or anti-CD3 loaded KT64/86 was determine using a xenogeneic model of GVHD (p≥0.05 for KT R2 and CD3/28R2 vs. PBMNC only, p=0.97 for KT vs. CD3/28).

FIG. 21 is a series of images demonstrating that cultured nTreg maintain Foxp3 expression and suppressive function after cryopreservation and thawing. Cultured nTregs were assayed for phenotype and suppressive function before and after freezing. (A) demonstrates that nTreg recovery after freezing and thawing was tested for three nTreg cultures (n=3 thaws each). (B) demonstrate that nTreg cultures maintain % CD4⁺ and % CD127⁻, Foxp3⁺ after freeze thaw. (C) demonstrates that cryopreserved nTregs maintain suppressive function (by CFSE assay) directly after thawing. Data for (B) and (C) are three independent experiments with a single assay on fresh cells, and three separate thaws for frozen/thawed.

FIG. 22 is is a series of images demonstrating that sort purified nTreg expand further than bead-purified nTreg. nTreg were purified in a two-step procedure whereby CD25+ cells were enriched using anti-CD25 magnetic beads, followed by sorting on CD4+25++127-Gating strategy (A), and representative example showing pre-and post-sort Foxp3 purity (B). Note that starting material for sort was PBMNC purified using anti-CD25 magnetic beads. (C) depicts expansion per stimulation for bead-vs. sort-purified nTreg with Rapamycin.

FIG. 23 depicts the phenotype of re-stimulated nTreg. Sort-purified nTreg were expanded with KT64/86 using 0-4 re-stimulations and stained for LAP (A), CD62L and CD27 (B), CCR7 (C) and (D), or CD45RA (E). Expression was assessed at 7 days after re-stimulation and after they returned to resting size, except for non-re-stimulated nTreg (R0) which is from day 17. Note all samples were from the same experiment (except for the longitudinal series in C) and were frozen, thawed, stained and analyzed concurrently so that direct comparisons could be made. (A) demonstrates that LAP expression is upregulated on Foxp3+ cells on day 7 after re-stimulation. (B) demonstrates that multiple re-stimulations cause nTreg to lose expression of CD62L and CD27. (C) demonstrates that CCR7 expression is induced by re-stimulation (Day 7), but decreases when cells return to resting size. (D) depicts time-course of CCR7 expression in R3 nTreg (re-stimulated on day 43) showing CCR7 expression spontaneously reappears ˜19 days after re-stimulation. (E) demonstrates that nTreg re-stimulation causes loss of CD45RA expression on day 7, but expression is restored before cells reach resting size. All data are CD4-gated and are representative of three experiments done with sorted nTreg, and similar data were observed with bead-purified nTreg.

FIG. 24 is an image depicting phenotypic changes in nTregs after re-stimulation. To assess changes in differentiation state resulting from multiple re-stimulations, PB nTreg stimulated once or a total of 5 times were stained for the following markers: CD27, CD45RA, CD57. Naïve cells were defined as CD27+45RA+57-, and memory cells, all other cells. In comparison with Tregs receiving a single stimulation (circle), re-stimulated nTregs (square) acquired a more differentiated state as shown by loss of CD27 expression in memory cells. These more mature nTregs did not acquire CD57 expression and therefore did not become senescent.

FIG. 25 is a series of images depicting T-cell receptor (TCR) Vβ usage of n Tregs before and after expansion. After repeated rounds of expansion, the T-cell receptor repertoire remained essentially unchanged with no significant skewing noted in the n Tregs after expansion. Each panel (A)-(C) depicts a separate sample comparing before and after TCR-Vβ usage. The Arden classification is used to depict the different TCR-Vβ family.

FIG. 26 is a series of images demonstrating that loss of Foxp3+ cells is due to increased cycling of Foxp3− cells. To differentiate between conversion of Foxp3+ to Foxp3− vs. outgrowth of Foxp3−, the relative proliferation of each population was monitored longitudinally using Ki-67. (A) FIG. 26A depicts a representative example of Ki-67 staining on Foxp3+vs. Foxp3− cells over time in cultures receiving a single re-stimulation in the absence of rapamycin−. (B) FIG. 26B depicts a comparison of Foxp3 expression vs. the relative ratio of proliferating Foxp3−: Foxp3+ cells over time demonstrating the decrease in Foxp3+ cells is concomitant with an increase in Foxp3− cycling.

FIG. 27 is an image demonstrating that nTreg stimulated once or a total of 5X suppress CD4 and CD8 responses equivalently. nTreg were purified from PB and were expanded with one or five rounds of stimulation using CD3-loaded/KT64/86. Cells were frozen after returning to resting size, thawed at the same time and suppressive function assessed by CFSE assay gated on either CD4+ or CD8+ cells. Results showing equivalent suppression of CD4+ and 8+ cells are representative of ≥5 experiments.

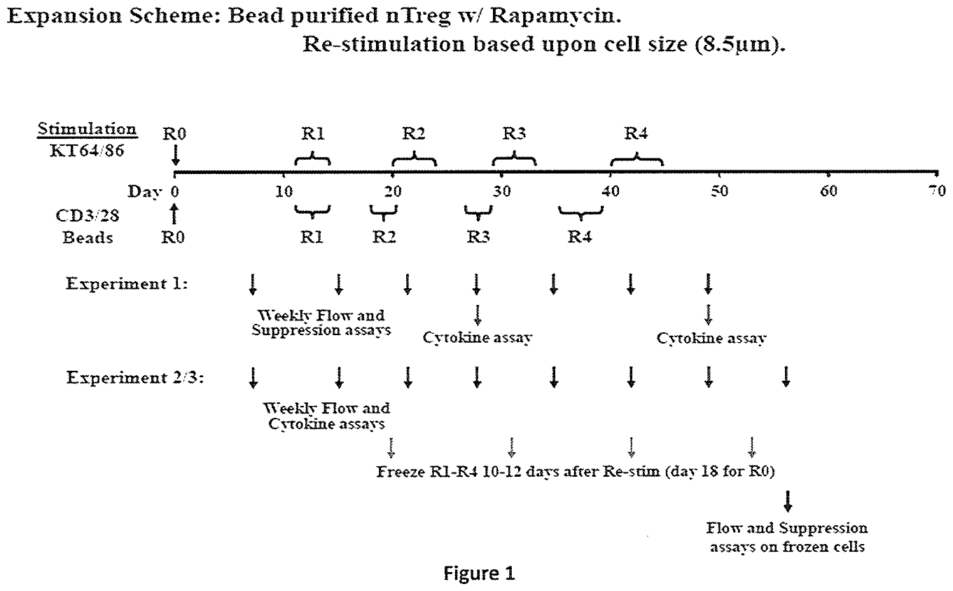

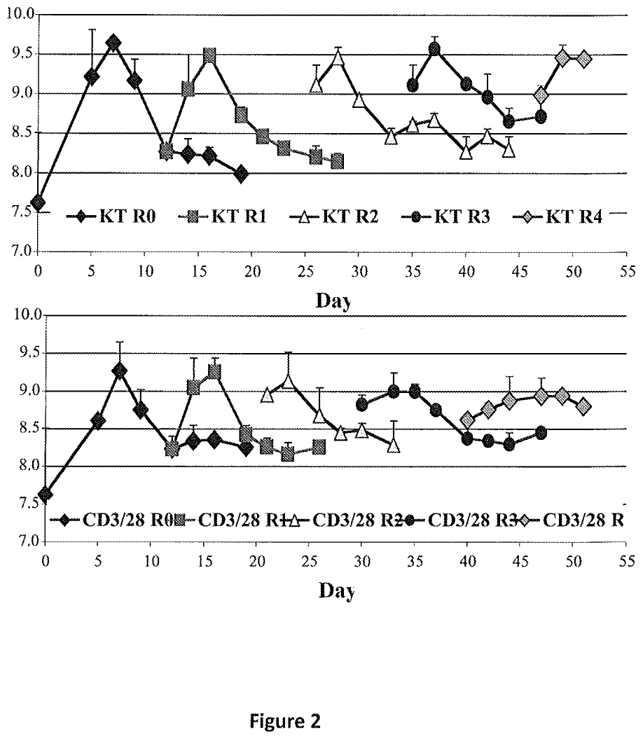

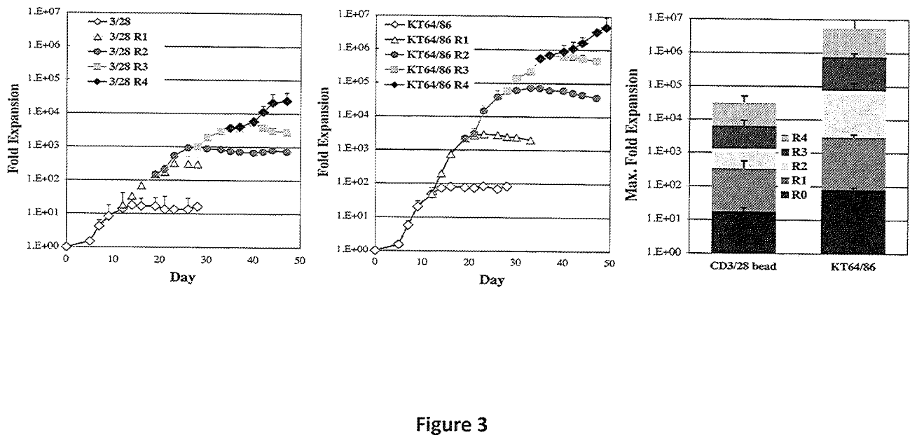

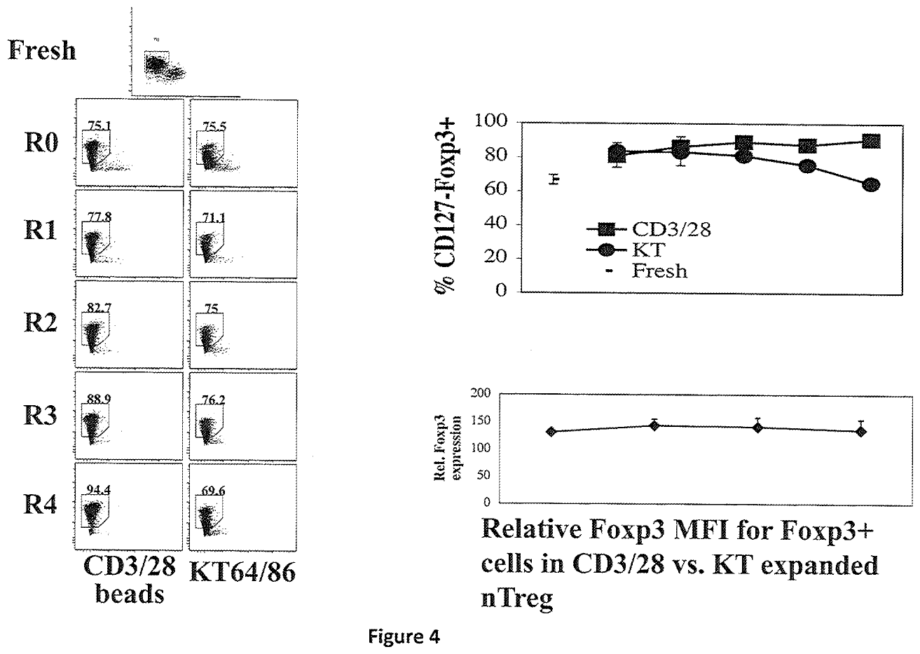

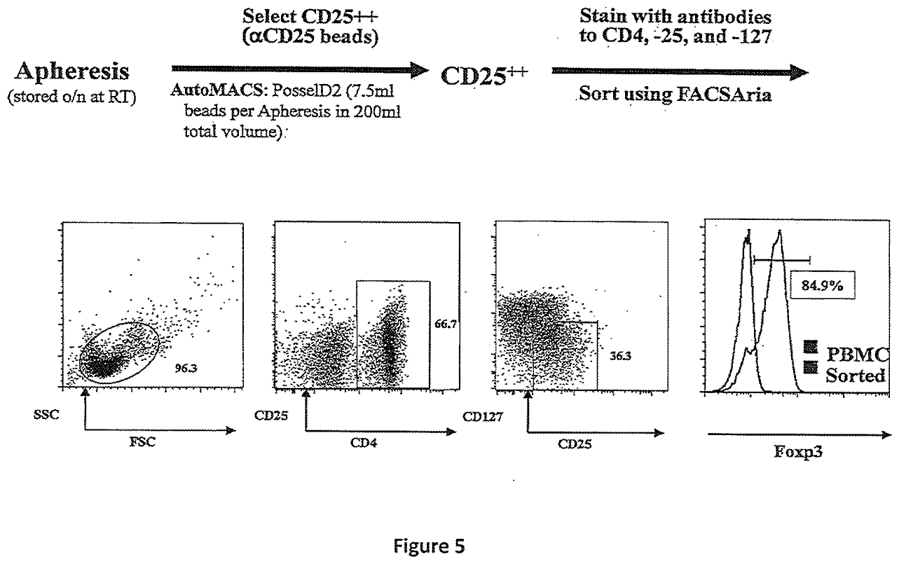

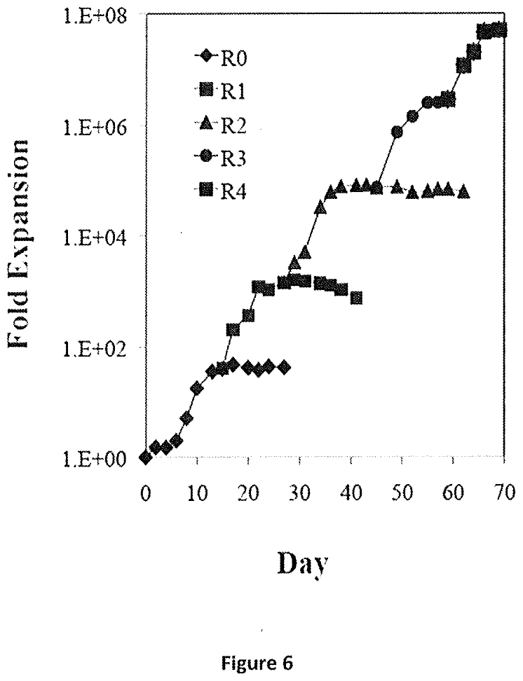

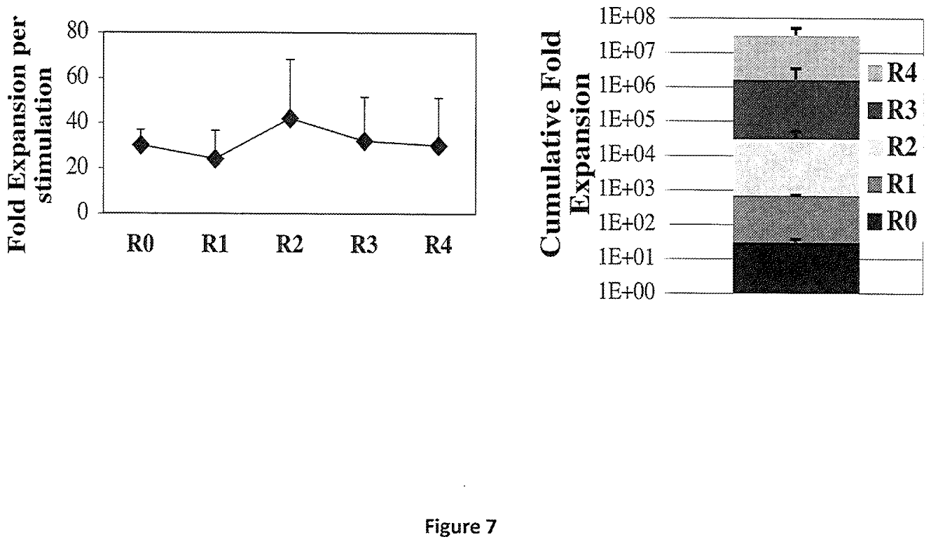

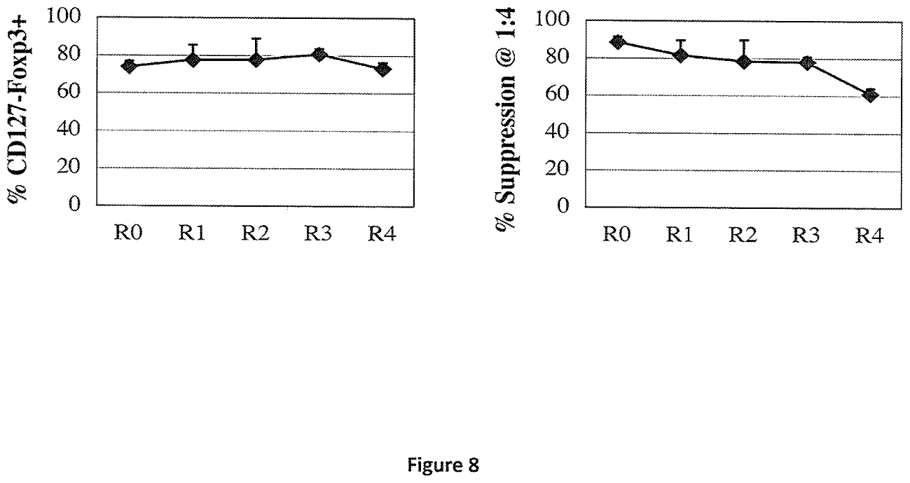

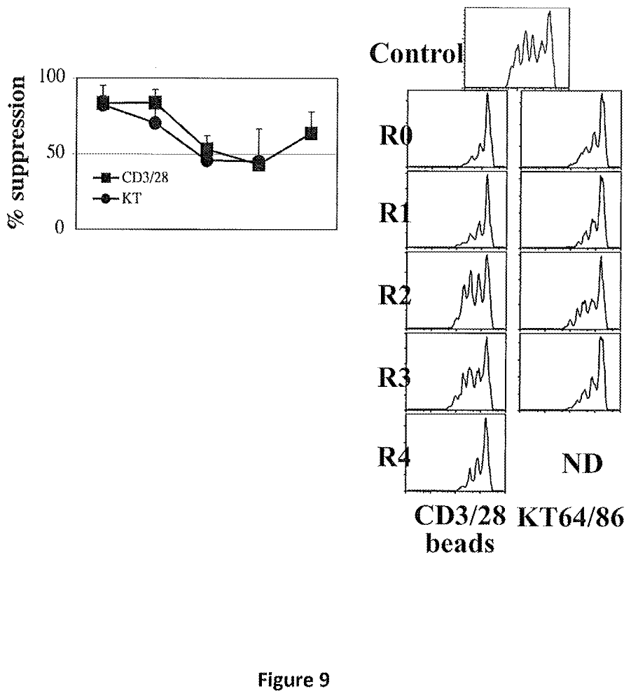

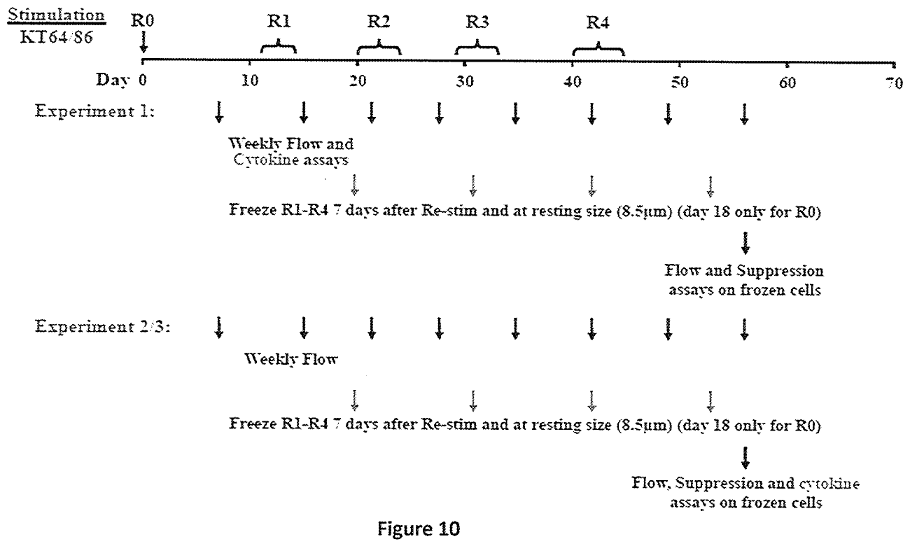

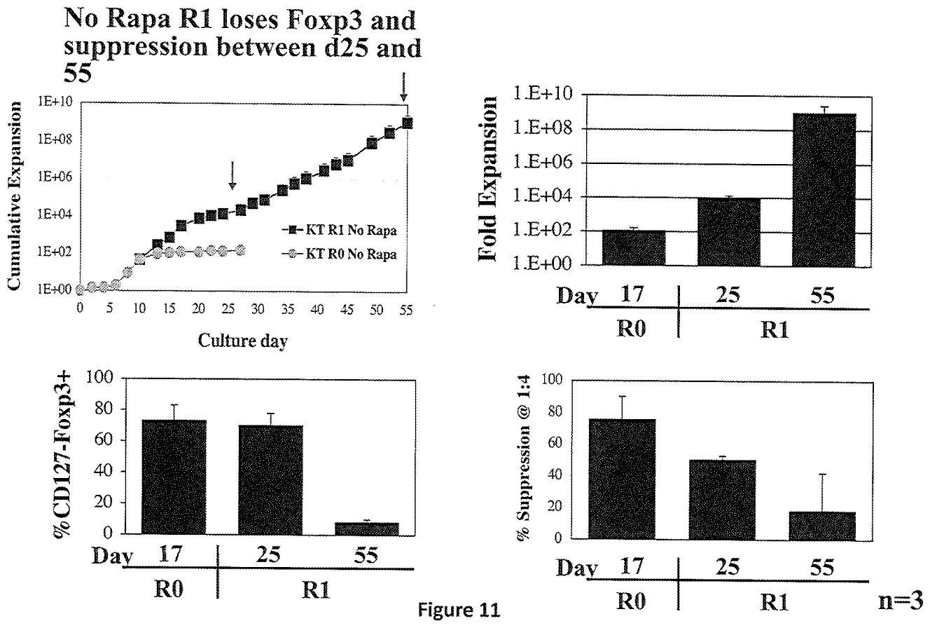

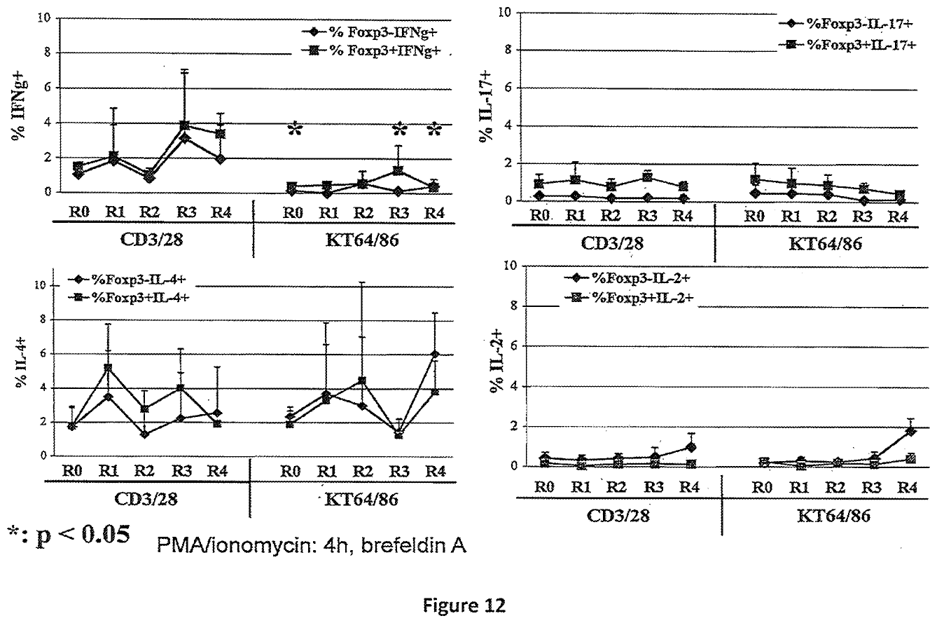

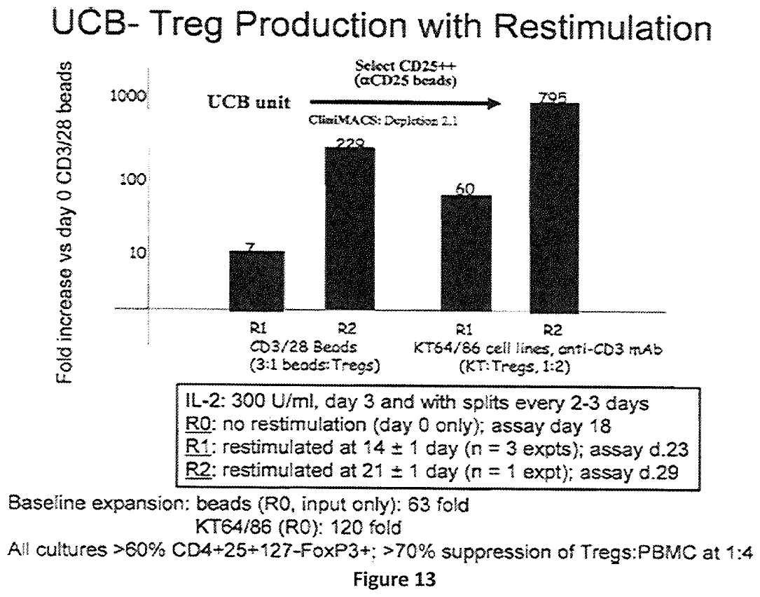

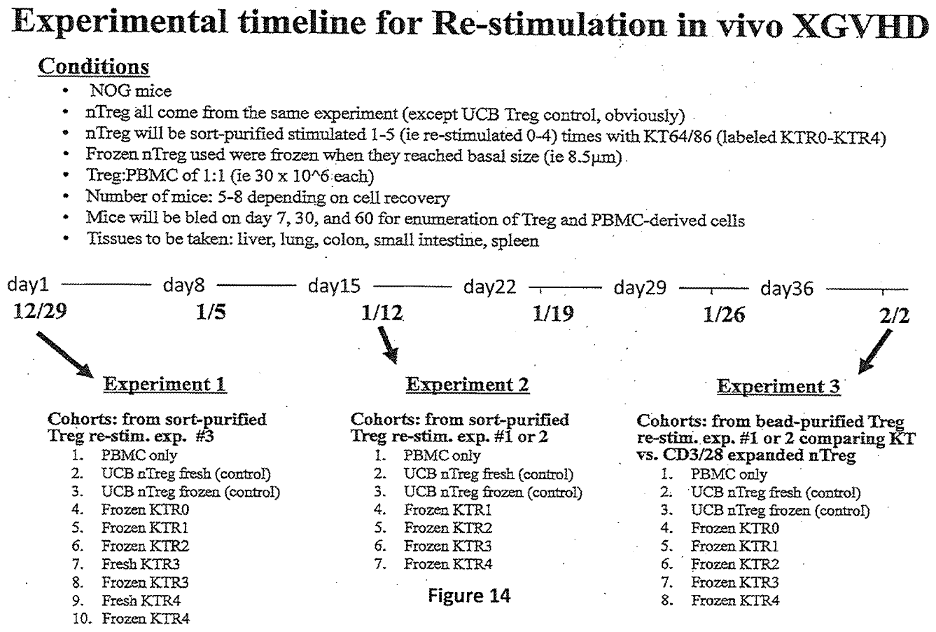

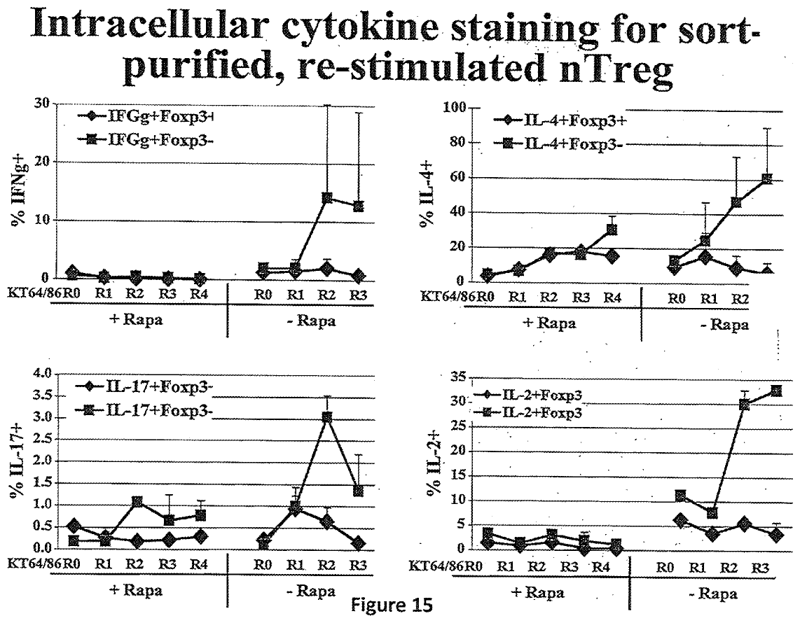

### DETAILED DESCRIPTION OF THE INVENTION

The present invention includes compositions and methods for expanding T regulatory cells (Tregs), preferably natural T regulatory cells (nTregs). More preferably, the expanded cells retain nTreg phenotype and suppression activity following expansion.

The invention provides compositions and methods for expanding Tregs, preferably nTregs, without the subsequent reversion of the nTregs to T effector cells. Accordingly, such an expansion methodology allows for the establishment of a cell bank.

In one aspect of the present invention, nTreg expansion can be performed by isolating nTregs from a desired cell source and subsequently culture expanding the cells in the presence of a primary signal and a co-stimulatory signal. Agents useful for stimulating a primary signal and an a co-stimulatory signal on Tregs may be used in soluble form, attached to the surface of a cell, or immobilized on a surface as described herein. In a preferred embodiment both primary and co-stimulatory agents are co-immobilized on a surface, for example a bead or an engineered cell. In one embodiment, the molecule providing the primary activation signal, such as a CD3 ligand, and the co-stimulatory molecule, such as a CD28 ligand are coupled to or loaded on the same surface, for example, a particle or an engineered cell.

In another embodiment, the invention provides a method of expanding Tregs, preferably nTregs to unprecedented numbers using a repetitive stimulation procedure. In one embodiment, the method of expanding nTregs comprises restimulating nTregs based upon cell size. Preferably, nTregs exhibiting a cell size about the size of a resting nTreg are chosen for restimulation. In some instances, the size of a resting nTreg is about 8.5 μm. That is, the invention is based on the discovery that cell size is a parameter that contributes to the success of expanding nTregs without losing nTreg phenotype and suppressor activity.

In another embodiment, the method of expanding nTregs comprises restimulating nTregs in the presence of Rapamycin. Preferably, nTregs isolated from peripheral blood is re-stimulated in the presence of Rapamycin. That is, the invention is based on the discovery that Rapamycin contributes to the success of expanding nTregs isolated from peripheral blood without losing nTreg phenotype and suppressor activity. Preferably, the expanded cells of the invention maintain Foxp3 profile indicative of n Tregs.

In one embodiment, the population of expanded nTregs expresses specific natural Treg markers such as Foxp3 and Latency Associated Peptide (LAP), displayed Treg specific demethylation in the Foxp3 gene, and contain very few IL-2, IFNγ, IL-17 secreting cells. The expanded cells of the invention also are able to suppress lethality in a xenogeneic model of GVHD.

In other embodiments, at least a portion of the active cell population is stored for later implantation/infusion. The population may be divided into more than one aliquot or unit such that part of the population of nTregs is retained for later application while part is applied immediately to the patient. Moderate to long-term storage of all or part of the cells in a cell bank is also within the scope of this invention.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy-or amino-terminus, can be modified by methylation, amidation, acetylation or substitution with other chemical groups which can change a peptide's circulating half life without adversely affecting activity of the peptide. Additionally, a disulfide linkage may be present or absent in the peptides.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “agent”, “ligand”, or “agent that binds a cell surface moiety”, as used herein, refers to a molecule that binds to a defined population of cells. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), a carbohydrate, or the like. Within the specification and in the context of T cell stimulation, antibodies and natural ligands are used as prototypical examples of such agents.

The terms “agent that binds a cell surface moiety” and “cell surface moiety”, as used herein, are used in the context of a ligand/anti-ligand pair. Accordingly, these molecules should be viewed as a complementary/anti-complementary set of molecules that demonstrate specific binding, generally of relatively high affinity.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of an mRNA molecule also consists of the nucleotide residues of the mRNA molecule which are matched with an anti-codon region of a transfer RNA molecule during translation of the mRNA molecule or which encode a stop codon. The coding region may thus include nucleotide residues corresponding to amino acid residues which are not present in the mature protein encoded by the mRNA molecule (e.g., amino acid residues in a protein export signal sequence).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 5′-ATTGCC-3′ and 5′-TATGGC-3′ share 50% homology.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “modulate” is meant to refer to any change in biological state, i.e. increasing, decreasing, and the like.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

“Quiescent”, as used herein, refers to a cell state wherein the cell is not actively proliferating.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

The term “subject” is intended to include living organisms in which an immune response can be elicited (e.g., mammals), preferably a human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division. By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

A “stimulatory molecule,” as the term is used herein, means a molecule on a T cell that specifically binds with a cognate stimulatory ligand present on an antigen presenting cell (e.g., an aAPC of the invention, among others).

“Loaded” with a peptide, as used herein, refers to presentation of an antigen in the context of an MHC molecule. “Loaded” as used herein also means the binding of an antibody to an Fc binding receptor on a cell, such as CD32 and/or CD64.

A “co-stimulatory signal”, as used herein, refers to a signal, which in combination with a primary signal, such as TCR/CD3 ligation, leads to T cell proliferation and/or upregulation or downregulation of key molecules.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

### DESCRIPTION

The present invention is based on the successful use of GMP-grade reagents to repeatedly stimulate Tregs, preferably nTregs. For example nTregs can be repeatedly stimulated using a cell-based aAPC to massively increase yields of nTregs while maintaining their suppressive function. Preferably, the expanded nTreg population expresses nTreg specific markers (e.g., Foxp3 and LAP), displayed nTreg specific demethylation in the Foxp3 gene, and contained very few IL-2, IFNγ, IL-17 secreting cells.

The present invention provides a system and method for culturing and expanding natural T regulatory cells (nTregs) without substantially sacrificing suppressor activity. The method includes expanding nTregs based on cell size and optionally in the presence of Rapamycin. By culturing and expanding nTregs according to the invention, nTregs can survive and increase in number without reverting to T effector phenotype. Accordingly, the present invention allows for expansion of any nTreg such as nTregs isolated from umbilical cord blood (UCB) or peripheral blood, and substantially increasing the number of nTregs available for subsequent use following expansion.

The invention relates to the surprising discovery that nTregs can be expanded to unprecedented levels while retaining nTreg phenotype and suppressor activity. In some instances, nTreg expansion is based upon re-stimulating cells having a desired cell size. Preferably, cells are re-stimulated when the cells return toward the size of resting nTregs. In some instances, a resting or otherwise quiescent nTreg has a mean diameter of about 8.5 μm, and upon stimulation, the nTreg mean diameter increases for a period of time and begin to subsequently decrease when the cells become quiescent. Accordingly, the invention includes a method of expanding nTregs based on cell size in the presence or absence of Rapamycin.

The present invention provides compositions and methods for their use to expand a nTreg as well as numerous therapeutic uses relating to expansion and stimulation of nTregs. The therapeutic aspect of the expanded nTregs of the invention is based on the observation that despite four repetitive re-stimulations and expansion of >50-million fold, fresh and cryopreserved nTregs each were capable of suppressing lethality in a xenogeneic model of GVHD. Accordingly, the expanded cells of the invention can be used in a clinical setting, for example for the prevention and/or treatment of GVHD following blood and marrow transplantation, organ rejection, and autoimmune disease.

In one embodiment, the invention provides compositions and methods for generating therapeutic amounts of nTregs isolated from peripheral or umbilical cord blood. Prior are methods are deficient in arriving at a large number of cells that would be useful in a clinical setting.

The present invention encompasses methods and kits for the isolation and expansion of nTregs. Preferably, the method of expanding nTregs according to the invention does not substantially change the activity of the expanded nTregs. In some instances, expansion of nTregs occurs without reversion to T effector cells. Such expansion allows for the establishment of a cell bank.

The unprecedented large number of nTregs expanded according to the present invention that exhibits suppressor function equivalent to freshly isolated nTregs enables further characterization of nTregs. In addition, an important utility of these cells is that they enable clinical testing because prior art methods are deficient in arriving at large enough numbers of nTregs for effective clinical testing. Suppressor cell lines can be useful for enhancing allograft tolerance induction or down-modulating autoimmune diseases.

The present invention further encompasses nTreg cells isolated and expanded without substantially decreasing the activity of the nTregs. In marked contrast to the prior art, the data disclosed herein demonstrates that nTreg cells expanded according to the invention retain the ability to suppress T cell proliferation and to suppress T cell activation-dependent cytokines. Further, the present data demonstrate that cord blood CD4+CD25+ cells can form potent suppressor cells after isolation and culture expansion. In one embodiment, nTregs isolated from umbilical cord can be expanded by repeated stimulation with CD3/28 where the nTregs so expanded do not revert to T effector cell phenotype.

In another embodiment, nTregs isolated from peripheral blood can be expanded by repeated stimulation in the presence of Rapamycin where the nTregs so expanded to not revert to T effector cell phenotype. In another embodiment, nTregs isolated from peripheral blood can be expanded by repeated stimulation in the presence of Rapamycin where the nTregs so expanded to not revert to T effector cell phenotype.

In one embodiment, the invention relates to a method for minimizing the growth of T effectors cells in cell populations during the expansion of nTreg cells. The resulting nTreg cells are particularly useful for treating immune diseases, such as graft versus host disease.

### Sources of T Cells

Prior to expansion, a source of T cells is obtained from a subject. Non-limiting examples of subjects include humans, dogs, cats, mice, rats, and transgenic species thereof. Preferably, the subject is a human. T cells can be obtained from a number of sources, including peripheral blood mononuclear cells, bone marrow, lymph node tissue, spleen tissue, umbilical cord, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood by lysing the red blood cells and depleting the monocytes, for example, by centrifugation through a PERCOLL™ gradient. Alternatively, T cells can be isolated from umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19 and CD56. Depletion of these cells can be accomplished using an isolated antibody, a biological sample comprising an antibody, such as ascites, an antibody bound to a physical support, and a cell bound antibody. Examples for methods of isolating Tregs and nTregs are disclosed in US 2005/0196386 and 2006/0062763, all of which are incorporated by reference as if set forth in their entirety herein.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

nTregs can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° ° C. at a rate of 1° per minute and stored in the vapor phase of a liquid nitrogen storage tank. Other methods of controlled freezing may be used as well as uncontrolled freezing immediately at −20° C. or in liquid nitrogen.

The population of nTreg cells may comprise CD4+ cells, CD25+ cells, and FoxP3+ cells. In one specific embodiment, the population of nTreg cells comprises CD4+CD25+FoxP3+ cells.

Expansion of nTregs

The present invention further comprises a method of multiplying, expanding or otherwise culturing a nTreg isolated using the methods disclosed herein or methods generally known in the art. As demonstrated by the data disclosed herein, multiplying a nTreg cell isolated by the methods of the present invention can be multiplied by about 10 fold, 100 fold, 1000 fold, 10,000 fold, 100,000 fold, 1,000,000 fold, 10,000,000 fold, or more using the methods disclosed herein. Following isolation, a nTreg cell is incubated in cell medium in a culture apparatus for a period of time or until the cells reach confluency before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. nTreg cell medium may be replaced during the culture of the nTreg cells at any time. Preferably, the nTreg cell medium is replaced about every 2 to 3 days. nTreg cells are then harvested from the culture apparatus whereupon the nTreg cells can be used immediately or cryopreserved to be stored for use at a later time. nTreg cells may be harvested by trypsinization, EDTA treatment, or any other procedure used to harvest cells from a culture apparatus.

In one aspect of the present invention, ex vivo nTreg expansion can be performed by isolation of nTregs and subsequent stimulation followed by further expansion based upon cell size. Preferably, the cells are re-stimulated based on cell size. More preferably, the cells are re-stimulated when the cells return toward the size of resting nTregs. The rate of T cell proliferation is monitored periodically (e.g., daily) by, for example, examining the size or measuring the volume of the T cells, such as with a Coulter Counter. In this regard, a resting or otherwise quiescent nTreg has a mean diameter of about 8.5 μm, and upon stimulation, the nTreg mean diameter increases for a period of time and begin to subsequently decrease when the cells become quiescent. When the mean T cell diameter decreases to approximately 8.5 μm, the nTregs may be re-stimulated to induce further proliferation of the T cells. In some instances, the nTregs are cultured in the presence of Rapamycin. Accordingly, the invention includes a method of expanding nTregs based on cell size in the presence or absence of Rapamycin.

The period of initial stimulation or restimulation as described herein (contact with agents as described herein) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The period of initial stimulation or restimulation as described further herein (contact with agents as described herein) can be longer, for example 1, 2, 3, 4, 5, 6, 7, 8, 9, 10, 11, 12, 13, 14, or more days. The cells of the invention can be re-stimulated for multiple rounds and yet retain natural Treg phenotype. For example, the cells of the invention can be re-stimulated for at least four rounds of restimulation.

In one embodiment of the invention, the T cells may be stimulated by a single agent. In another embodiment, T cells are stimulated with two agents, one that induces a primary signal and a second that is a co-stimulatory signal. Ligands useful for stimulating a single signal or stimulating a primary signal and an accessory molecule that stimulates a second signal may be used in soluble form, attached to the surface of a cell or immobilized on a surface as described herein. In a preferred embodiment both primary and secondary agents are co-immobilized on a surface, for example a bead or an artificial presenting cell (aAPC). In one embodiment, the molecule providing the primary activation signal, such as a CD3 ligand, and the co-stimulatory molecule, such as a CD28 ligand are coupled to or loaded on the same surface, for example, a particle or an aAPC.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

The medium used to multiply the nTreg cells of the present invention comprises an agent that can stimulate CD3 and CD28 on the nTreg cell. For example, an agent that can stimulate CD3 is an antibody to CD3, and an agent that can stimulate CD28 is an antibody to CD28. In some instances, the medium further comprises Rapamycin. This is because, as demonstrated by the data disclosed herein, a cell isolated by the methods of the present invention can be multiplied approximately 10 fold, 100 fold, 1000 fold, 10,000 fold, 100,000 fold, 1,000,000 fold, 10,000,000 fold, or more by culturing the cell with an antibody that binds CD3, and antibody that binds CD28, and optionally Rapamycin, without losing nTreg phenotype and suppressor function.

In one aspect of the present invention, ex vivo nTreg expansion can be performed by isolation of T cells and subsequent stimulation followed by further expansion based upon cell size. In one embodiment of the invention, the T cells may be stimulated by a single agent. In another embodiment, T cells are stimulated with two agents, one that induces a primary signal and a second that is a co-stimulatory signal. Ligands useful for stimulating a single signal or stimulating a primary signal and an accessory molecule that stimulates a second signal may be used in soluble form, attached to the surface of a cell or immobilized on a surface as described herein. In a preferred embodiment both primary and secondary agents are co-immobilized on a surface, for example a bead or an artificial presenting cell (aAPC). In one embodiment, the molecule providing the primary activation signal, such as a CD3 ligand, and the co-stimulatory molecule, such as a CD28 ligand are coupled to or loaded on the same surface, for example, a particle or an aAPC.

In some instances, expansion of nTregs is based on cell size. This is because, as demonstrated by the data disclosed herein, cell size guided restimulation dramatically increased fold expansion of nTregs in a medium comprising an agent that can stimulate CD3 and CD28 on the nTreg cell.

An example of an agent that can stimulate CD3 is anti-CD3 antibody; an agent that can stimulate CD28 is an anti-CD28 antibody. In one embodiment, the antibodies of the present invention are conjugated or otherwise attached to a bead, such as a magnetic bead or a Dynal bead. Such beads are known in the art and are described elsewhere herein. In another embodiment, the antibodies are presented on an artificial presenting cell (aAPC).

Accordingly, in certain aspects, the present invention includes expanding nTregs in the presence of an aAPC. The extensive disclosure regarding aAPCs provided in WO 03/057171, US2003/0147869, US2006/0034810, 2004/0101519 are incorporated by reference as if set forth in their entirety herein. However, the present invention is based on the surprising discovery that expansion of nTregs by re-stimulating nTregs based on cell size dramatically increases fold expansion with maintaining nTreg phenotype. In some embodiments, the nTregs are expanded in the presence of Rapamycin.

In the context of an aAPC, a primary signal, usually mediated via the T cell receptor/CD3 complex on a T cell, initiates the T cell activation process. Additionally, numerous co-stimulatory molecules present on the surface of a T cell are involved in regulating the transition from resting T cell to cell proliferation. Such co-stimulatory molecules, also referred to as “co-stimulators”, which specifically bind with their respective ligands, include, but are not limited to, CD28 (which binds with B7-1 [CD80], B7-2 [CD86]), PD-1 (which binds with ligands PD-L1 and PD-L2), B7-H3, 4-1BB (binds the ligand 4-1BBL), OX40 (binds ligand OX40L), ICOS (binds ligand ICOS-L), and LFA (binds the ligand ICAM). Thus, the primary stimulatory signal mediates T cell stimulation, but the co-stimulatory signal is then required for T cell activation, as demonstrated by proliferation.

A skilled artisan would understand, based upon the disclosure provided herein, that an aAPC comprising an antibody can be produced, as exemplified elsewhere herein, by introducing a nucleic acid encoding a human Fcγ receptor (e.g., CD32 or CD64), into the aAPC. The CD32 and/or CD64 expressed on the aAPC surface can then be “loaded” with any desired antibody that binds with CD32 and/or CD64, including, but not limited to, antibody that specifically binds CD3 and antibody that specifically binds with CD28.

### Cell Populations

T helper cells (also known as effector T cells or Th cells) are a sub-group of lymphocytes (a type of white blood cell or leukocyte) that plays an important role in establishing and maximizing the capabilities of the immune system and in particular in activating and directing other immune cells. Different types of Th cells have been identified that originate in outcome of a differentiation process and are associated with a specific phenotype. Following T cell development, matured, naive (meaning they have never been exposed to the antigen to which they can respond) T cells leave the thymus and begin to spread throughout the body. Naive T cells can differentiate into a T-helper 1 (Th1), T-helper 2 (Th2), T-helper 17 (Th17), regulatory T cell (Treg) or natural Treg phenotype.

Each of these Th cell types secretes cytokines, proteins or peptides that stimulate or interact with other leukocytes, including Th cells. However, each cell type has a peculiar phenotype and activity that interferes and often conflict with the other.

nTregs are a component of the immune system that suppresses biological activities of other cells associated to an immune response. In particular, nTregs can secrete immunosuppressive cytokines TGF-beta and Interleukin 10, and are known to be able to limit or suppress inflammation.

The present invention is based on the discovery that nTregs can be expanded without the subsequent reversion to T effector cells. That is, the methods of the invention allows for expanding nTregs without substantial loss of suppressor activity.

The nTreg of the present invention further comprises certain antigenic markers, some of which are present when a nTreg cell is isolated from an umbilical cord blood sample, some of which are present when the nTreg cell is multiplied, cultured, or otherwise expanded according to the methods of the present invention. Such antigenic markers are useful in the identification of a nTreg cell of the present invention, and allow one of skill in the art to determine if a nTreg cell isolated and multiplied according to the methods of the present invention has the properties and biological activities of a nTreg cell of the present invention. Such biological activities include, but are not limited to, suppression of an allogeneic immune response, inhibition of cytokine accumulation in an immune response accompanied by less inhibition of chemokine production, the production of IL-2, IL-10 and gamma interferon, the expression of TGF-beta latency associated protein (LAP), and suppressor activity independent of IL-10 and TGF-beta. Markers on the nTreg cell of the present invention include, but are not limited to, CD25, CD4, CTLA4, CD27, CD26L and Fox P3.

### Therapy

The present invention further comprises a method for inhibiting proliferation of a T cell. Such inhibition can occur in vitro or in vivo, preferably in an animal, more preferably in a mammal, even more preferably in a human. This is because, as demonstrated by the data disclosed herein, HLA mismatched T cells in a mixed lymphocyte reaction (MLR) were inhibited from proliferating by a factor greater than about 95% in the presence of a nTreg cell isolated and expanded according to the methods of the present invention. Further, as demonstrated by the data disclosed herein, nTreg cells isolated and expanded according to the methods of the present invention are potent suppressors of T cell proliferation at ratios of from about 1:16 to about 1:32 (nTreg:T cell). Further, the nTreg cells of the present invention are active in suppressing an immune response when a antigen presenting cell, such as a dendritic cell, is mature and activated. Thus, the cells of the present invention can be used to inhibit active immune responses or to prevent an immune response.

The method of the present invention comprises contacting a T cell with a nTreg cell isolated and expanded according to the methods of the present invention such that the proliferation of a T cell is inhibited. The nTreg cell can be administered using techniques well known in that art so that a nTreg contacts, or is in proximity, to an immune cell, such as a T cell, dendritic cell, plasma cell, and the like.

nTregs expanding according to the methods of the present invention are uniform and potent suppressor cells. Further, the expanded nTreg cells of the present invention can be administered to an animal, preferably a mammal, even more preferably a human, to suppress an immune reaction, such as those common to autoimmune diseases such as diabetes, psoriasis, rheumatoid arthritis, multiple sclerosis, GVHD, enhancing allograft tolerance induction, transplant rejection, and the like. In addition, the cells of the present invention can be used for the treatment of any condition in which a diminished or otherwise inhibited immune response, especially a cell-mediated immune response, is desirable to treat or alleviate the disease.

The nTregs generated according to the present invention can also be used to treat autoimmune diseases. Examples of autoimmune disease include but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenia purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

The nTregs generated according to the present invention can also be used to treat inflammatory disorders. Examples of inflammatory disorders include but are not limited to, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

Cells of the invention can be administered in dosages and routes and at times to be determined in appropriate clinical trials. Cell compositions may be administered multiple times at dosages within these ranges. The cells of the invention may be combined with other methods. The cells of the invention for administration may be autologous, allogeniec or xenogenic to the patient undergoing therapy.

The administration of the cells of the invention may be carried out in any convenient manner. The cells of the present invention may be administered to a patient subcutaneously, intradermally, intramuscularly, by intravenous (i.v.) injection, or intraperitoneally. In some instances, the cells of the invention are administered to a patient by intradermal or subcutaneous injection. In other instances, the cells of the invention are administered by intravenous injection. In other instances, the cells of the invention are injected directly into a tumor or lymph node.

The cells of the invention can also be administered using any number of matrices. The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized, as such these patents are incorporated herein by reference in their entirety. Matrices comprise features commonly associated with being biocompatible when administered to a mammalian host. Matrices may be formed from natural and/or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples therefore, specifically point out the preferred embodiments of the present invention, and are not to be construed as limiting in any way the remainder of the disclosure.

### Example 1: Expansion of Umbilical Tregs

Naturally occurring CD25+CD4+ suppressor cells (Tregs) cells play an active part in establishing and maintaining immunological unresponsiveness to self constituents (i.e., immunological self tolerance) and negative control of various immune responses to non-self antigens. There are a paucity of reliable markers for defining Tregs, but naturally occurring CD25+CD4+ Tregs are the most widely studied because accumulating evidence indicates that this population plays a crucial role in the maintenance of immunological self tolerance and negative control of pathological as well as physiological immune responses. Their natural presence in the immune system as a phenotypically distinct population makes them a good target for designing ways to treat or prevent immunological diseases and to control pathological as well as physiological immune responses. However, little, if any methods exist to expand and manipulate this population of cells.

Tregs have the potential to be used in therapy for graft versus host disease (GVHD), and as an adjunct therapy in transplant rejection. The current methods for expansion of Tregs lead eventually to the reversion of the cells to T effector cells which may exacerbate rather than modulate the immune response in GVHD. The following experiments were designed to expand Tregs without the subsequent reversion to T effector cells.

Tregs have been shown to be present in cord blood. CD25+ and CD25− CD4+ T-cells were isolated from umbilical cord blood. Cord blood mononuclear cells were prepared by centrifugation over Ficoll-Hypaque according to the manufacturer's directions. After CD34+ depletion with magnetic microbeads (Miltenyi Biotec, Auburn, Calif.), CD25+ cells were isolated by positive selection with directly conjugated anti-CD25 magnetic microbeads (4 microliters per 10⁷ cells; Miltenyi Biotec). Cells were then applied to a second magnetic column, washed, and re-eluted. After the double column procedure cells were routinely >90% pure (for CD4/CD25) by FACS analysis. The non-CD25 fraction was then applied to another magnetic column to deplete any remaining CD25+ cells, before isolation of CD4+CD25− cells by positive selection with anti-CD4 mAb-coated microbeads (Miltenyi Biotec). Stringent purification of adult CD25+ cells used anti-CD25-FITC and anti-FITC microbeads (2 microliters per 10⁷ cells), and passage over magnetic column and elution for two cycles. This was followed by releasing the magnetic beads, and subsequent lineage depletion with anti-CD8, CD14, CD19, and CD56 direct conjugated microbeads (CD4+CD25++ lin-) as described in Godfrey, et al. (2005, Blood 105: 750-758).

Briefly, umbilical cord blood was used as a source of T-regs. Treg were isolated as described elsewhere herein and expanded in vitro by repeated stimulation, based upon cell size, with CD3/28 beads (e.g., 3:1 beads: Tregs) or KT64/86 loaded with anti-CD3 antibody (1:2 KT:Tregs). CD25 cells were cultured in X-Vivo-15 (BioWhittaker, Walkersville, MD) media supplemented with 10% human AB serum (ValleyBiomedical, Winchester, VA), L-glutamine (Invitrogen), and N-acetylcysteine (American Regent, Shirley, NY). Recombinant IL-2 (300 IU/mL; Chiron, Emeryville, CA) was added on day 3 and maintained for culture duration. It was observed that the expanded cells did not revert to T effector cells. Such reversion would normally be expected in Tregs obtained from other sources.

### Example 2: Expansion of Adult Peripheral Blood Using Rapamycin

Peripheral blood mononuclear cells (PBMC) were isolated from buffy coat preparations, which were derived from the whole blood of normal healthy volunteer donors (Memorial Blood Centers, Minneapolis, Minn.). Leukocyte rich buffy coat cells were centrifuged over Ficoll-Hypaque layers to collect PBMC. CD25+ cells were isolated using the following indirect antibody based microbeads. PBMC were stained with anti-CD25-FITC, washed and then bound secondarily to anti-FITC multi-sort microbeads (5 microliters/10⁷ cells, Miltenyi Biotec, Auburn, Calif.) and positively selected. The CD25+ cells were reapplied to a second column, washed and re-eluted. After column purification, the anti-FITC multisort beads were detached. The CD25+ cells were further depleted of CD8, CD14, CD19, CD20, and CD56 expressing cells with a cocktail of mAb-coated microbeads for lineage depletion Godfrey, et al. (2004, Blood 104: 453-461). These CD25+ lineage depleted cells were then selected for CD45RA by direct positive selection with anti-CD45RA microbeads (20 microliters/10⁷ cells, Miltenyi). In some cases, a further purification of anti-HLA-DR+ cells was isolated from the CD45RA⁻ cells by positive selection with anti-HLA-DR microbeads (20 microliters/10⁷ cells, Miltenyi). The CD25 cells were further depleted of CD25 by a second round of depletion with direct anti-CD25 microbeads (20 microliters/10⁷ cells, Miltenyi). After CD25 depletion these cells were then positively selected for CD4 with direct anti-CD4 microbeads (20 microliters/1010⁷ cells, Miltenyi).

To improve on the purification strategy, CD25+ cells were analyzed for subsets by flow cytometric analysis. A subset of CD4+CD25+ cells could be further enriched for suppressor cells, or conversely, a subset might be enriched for conventional T cells.

Experiments were designed to expand Tregs isolated from adult peripheral blood. Tregs were isolated by a two step procedure in which CD4 cells were first enriched by depleting CD8, −14, and −19+ cells with GMP-grade mAb coated microbeads, followed by positive selection of CD25++ cells using GMP-grade anti-CD25 microbeads (either 75 μl/2×10⁸ cells, Miltenyi). Treg were subsequently expanded in vitro by repeated stimulation with CD3/28 beads or KT64/86 loaded with anti-CD3 antibody. In addition, the expansion scheme involved expanding Tregs in the presence of Rapamycin (109 nM) and restimulation based upon cell size. It was observed that repeatedly stimulation in the presence of Rapamycin lead to a population of Tregs that did not revert to T effector cells.

### Example 3: Massive Ex Vivo Expansion of Human Natural Regulatory T Cells (nTregs) with Minimal Loss of In Vivo Functional Activity

Graft-versus-host disease (GVHD) is a frequent and severe complication following hematopoietic cell transplantation. Natural CD4⁺25⁺⁺ regulatory T-cells (nTregs) have proven highly effective in preventing autoimmunity and graft-versus-host disease (GVHD) and autoimmunity in murine models. Clinical application of nTreg has been severely hampered by their low frequency and unfavorable ex vivo expansion properties. Previously, it was demonstrated that umbilical cord blood (UCB) nTreg could be purified and expanded in vitro using GMP reagents. Recently, the first phase I clinical trial testing the safety of expanded nTreg and their capacity to suppress GVHD following UCB transplantation was completed. Since the initial number of nTreg in UCB units is limited, and average yield after expansion was only 1×10⁹ nTreg, experiments were designed to explore whether yield could be increased by purifying and expanding cells from peripheral blood (PB), which contains far larger quantities of nTreg.

The results presented herein demonstrate that PB nTregs were purified under GMP conditions and expanded 80-fold to yield to 19×10⁹ cells by culturing with Rapamycin, IL-2 and anti-CD3 antibody loaded, cell-based artificial antigen presenting cells (aAPCs) expressing the high affinity Fc receptor and CD86. A single re-stimulation increased expansion another 36-fold (to ˜3,000-fold total) and yield to >600×10⁹, while maintaining FoxP3 expression and in vitro suppressor function. nTreg expansion was increased an average of 44 million-fold when flow-sort purified nTreg were re-stimulated four times with aAPCs. Cryopreserved donor nTregs re-stimulated four times significantly reduced GVHD lethality induced by the infusion of human T-cells into immune deficient mice. The capability to efficiently produce donor cell banks of functional nTreg could revolutionize the treatment of GVHD and autoimmunity by providing a readily available, cost-effective and proven cellular therapy.

The materials and methods employed in these experiments are now described.

### Materials and Methods

### Treg Isolation and Culture

For all experiments, non-mobilized peripheral blood Leukapheresis products were collected from normal adult volunteers using FDA approved/cleared apheresis instruments. Written informed consent was obtained from all subjects. nTreg were purified using GMP magnetic beads or by sorting.

For bead-based nTreg purification, CD4+ T cells were enriched by MACS (all beads from Miltenyi Biotec, Auburn, CA) by depleting non-CD4s with GMP-grade mAb-coated microbeads (cocktail of CD8, CD14, CD19±CD56, 7.5 ml each/Apheresis product) in combination with a CliniMACS (Depletion 2.1-Max TNC=2.0×10¹⁰). Unbound cells were washed and CD25 Tregs were subsequently purified by positive selection using GMP-grade anti-CD25 microbeads (7.5 ml/Apheresis product) using CliniMACS (Enrichment 3.2). CD8−/CD14−/CD19−/CD25− cells were subsequently enriched for CD3+ feeder cells using GMP-grade anti-CD3 microbeads. All bead incubations were carried out as specified by the manufacturer (i.e. 30 minutes at RT for GMP-grade beads). All washes were performed at 300 x for 10 minutes at room temp.

nTregs were sort purified from peripheral blood mononuclear cells (PBMNC; Ficoll-Hypaque, Amersham Biosciences, Uppsala, Sweden) in a two-step procedure in which CD25+ cells were initially enriched from PBMNC by AutoMACS (PosselD2) with GMP-grade anti-CD25 microbeads (75 μl/2×10⁸ cells). CD25++ cells were stained with CD4, CD8, CD25 and CD127 and sorted via FACSAria as CD4+, CD8−, CD25++, CD127−. Note that the bead bound and fluorochome conjugated anti-CD25 antibodies recognize different epitopes.

Purified CD4+CD25+ cells were cultured with either GMP anti-CD3/CD28 coated Dynabeads (3:1 bead:cell) or with K562 cell lines engineered to express CD86 and the high affinity Fc Receptor (CD64) (2:1 nTreg:KT), that had been irradiated with 10,000 cGray and incubated with anti-CD3 (Orthoclone OKT3, Janssen-Cilag). In some experiments, nTregs were stimulated with KT64/86 that were pre-loaded, irradiated and frozen (1:1 nTreg:KT). Irradiated feeder cells (2600 rads, CD8-/CD14−/CD19−/CD25-/CD3+) were added to CD3/28 bead cultures at 1:1 feeder: Treg. nTregs were cultured in X-Vivo-15 media (BioWhittaker, Walkersville, MD) supplemented with 10% human AB serum (Valley Biomedical, Winchester, VA), GlutaMAX (Gibco) and N-acetylcysteine (USP). Recombinant IL-2 (300 IU/ml, Chiron, Emeryville, CA) was added on day 2 and maintained for culture duration. Cultures were maintained at 0.3-0.5×10⁶ viable NC/ml every 2-3 days.

Where indicated, Rapamycin (Rapammune, Wyeth-Ayerst) at 109 nM was added on day 0 and with subsequent media supplementation. Cell size and viability were determined by ViCell (Beckman Coulter).

Cryopreservation and Thawing of nTregs

To freeze nTregs, cultures were washed twice and resuspended at 50×10⁶/ml in X-Vivo 15. An equal volume of cold 2× freezing medium (80% human AB serum+20% DMSO) was added to the cells, and 25×10⁶ cells (i.e. 1 ml) were aliquoted into pre-chilled cryotubes (Nunc) which were immediately transferred to a rate controlled freezer. After freezing, samples were stored in liquid nitrogen. nTreg were thawed by incubating cryotubes at 37° C. until liquid was visible around a solid ice core. Cells were diluted in pre-warmed supplemented X-Vivo 15 (10 ml), layered onto a cushion of 200 μl 25% Human serum Albumin (CSL Behring), and centrifuged at 300 g for 10 minutes. Cells were collected from the interface and washed once more before use.

### Intracellular Cytokine Staining

Fresh or frozen nTreg were cultured in supplemented X-Vivo 15 for 4 hours±PMA (2 pg/ml) and Ionomycin (1 μg/ml) in the presence of Brefeldin A (100 ng/ml) (all Sigma). Frozen/thawed samples were cultured for 1 hour at 37° C. prior to re-stimulation. Cells were then harvested and stained for CD4, 25, Foxp3 and cytokine (IL-2, IL-4, IL-17, and IFNγ) or Granzyme B using the standard Foxp3 intracellular staining kit

### Flow Cytometry and Antibodies

Human-specific antibodies used for flow cytometry CD4 (RPA-T4), CD8 (RPA-T8), CD14 (MSE2), CD19 (HIB19), CD25 (M-A251), CD27 (M-T271), CD45RA (HI100), CD62L (Dreg56), Granzyme B and Ki-67 were purchased from BD Pharmingen. Antibodies to HLA-DR, CCR7, IFNg, IL-2, IL-4, IL-17 were from eBioscience, while Foxp3 (clone 249D) is from BioLegend and LAP (FAB2463P) is from R&D Systems Minneapolis, MN. Acquisition was performed using a FACScalibur or LSRII (BD Bioscience) and data were analyzed using FlowJo software (Tree Star Inc.).

### Suppression Assays

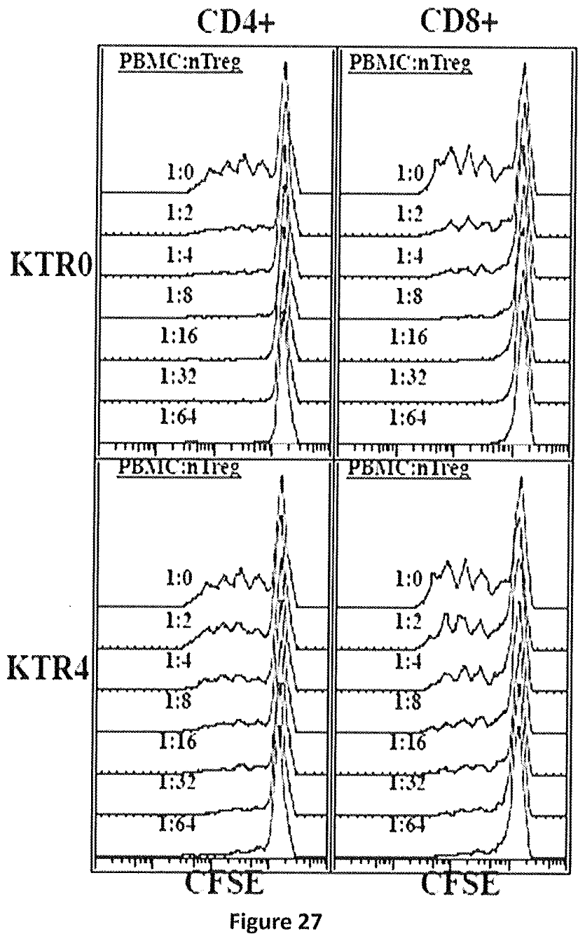

The in vitro suppressive capacity of expanded nTreg was assessed with a 5-carboxyfluorescein diacetate succinimide ester (CFSE) inhibition assay. Briefly, PBMNC were purified, labeled with CFSE (In Vitrogen), and stimulated with anti-CD3 mAb-coated beads (Dynal)±cultured nTreg (1:2-1:32 nTreg:PBMNC). On day 4, cells were stained with antibodies to CD4 and 8. Proliferation was analyzed using FlowJo (8.8.7), and suppression was determined from the Division Index (Treestar, Ashland, OR). It was observed that nTreg suppressed CD4 and 8 responses equivalently (FIG. 27).

### TSDR Methylation Status

To determine the methylation status of the Foxp3 gene, DNA was purified from frozen aliquots of bead-purified nTreg expanded with 3 or 4 re-stimulations using KT64/86. Samples were submitted to EpigenDx Inc. (Worcester, MA 01606) for bisulfite modification and sequencing of 11 CpG motifs in the TSDR. The standard deviation for methylation across all 11 sites was <11%.

VB Usage and Differentiation Status of Expanded nTreg

Cells were thawed and rested for 2 hours at 37° C. in RPMI 1640 supplemented with 10% heat-inactivated fetal calf serum, 100 U/mL penicillin G, 100 μg/ml streoptomycin sulfate, and 1.7 mM sodium glutamine. For the phenotypic analysis, cells were then washed with PBS and stained with a violet amine viability dye (Invitrogen) along with the following directly conjugated antibodies: anti-CD3 APC-H7 (BD Biosciences), anti-CD4 APC (BD Biosciences), anti-CD8 quantum dot (QD) 655, anti-CD27 PE-Cy5 (Beckman Coulter), anti-CD45RA QD705, and anti-CD57 FITC (BD Biosciences).

For the TCR Vb analysis, cells were transferred in 200 mL aliquots to a 96-well ‘V’ bottom microtiter plate containing each TCR Vb antibody, a violet amine viability dye (Invitrogen) along with the following directly conjugated antibodies: anti-CD3 APC-H7 (BD Biosciences), anti-CD4 APC (BD Biosciences), and anti-CD8 QD655. All QD conjugates were prepared in-house. After 30 minutes at room temperature, cells were washed with PBS containing 1% bovine serum albumin and 0.1% sodium azide, and then fixed with PBS containing 1% paraformaldehyde. Cells were analyzed using a modified LSRII flow cytometer (BD Immunocytometry Systems) equipped for the detection of 18 fluorescent parameters. Electronic compensation was conducted with antibody capture beads (BD Biosciences) stained separately with each individual antibody. Data analysis was performed using FlowJo software, version 8.6 (TreeStar).

### Xenogeneic GVHD Model

Briefly, T-, B- and NK-deficient NOD/Scid/yc−/− mice (Jackson) were housed in a pathogen-free facility in micro-isolator cages and on day 0, human PBMNCs (30×10⁶) were injected with or without expanded nTregs at either 1:1 or 1:2 (i.e. 30- or 15×10⁶ nTreg). Mice were assessed for signs of GVHD daily and weighed thrice weekly. To document PBMNC-associated T cell expansion, the animals were bled (10-40 μl), and red blood cells lysed. T cells derived from Treg or PBMNC were enumerated by Flow Cytometry by staining with antibodies to human CD4, CD8, HLA-A2 and CD45 and acquiring with a known number of counting beads (Sigma).

### Statistical Analysis

Data were analyzed by ANOVA or student's t-test. Probability (P) values ≤0.05 were considered statistically significant.

The results of the experiments are now described.

PB nTreg can be Purified and Expanded Using GMP Reagents and Protocols

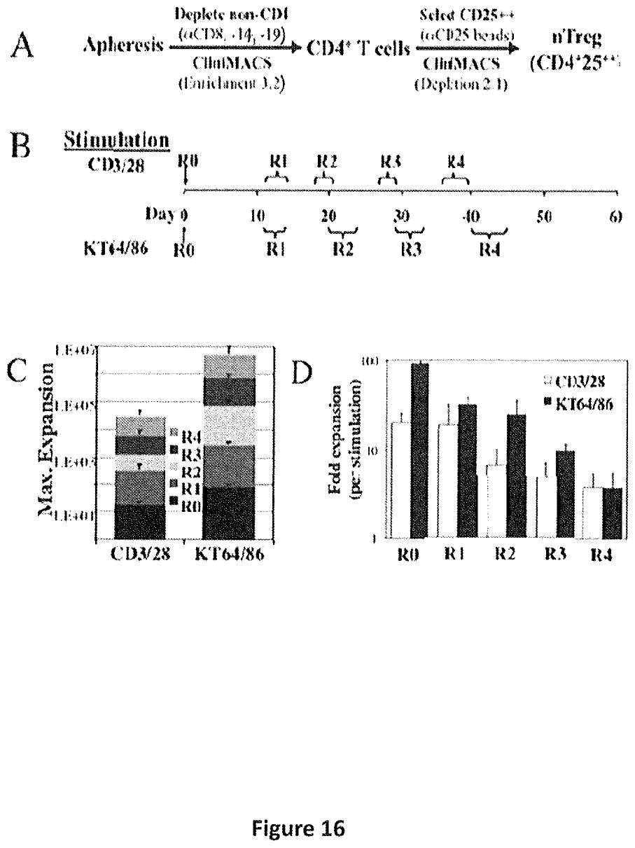

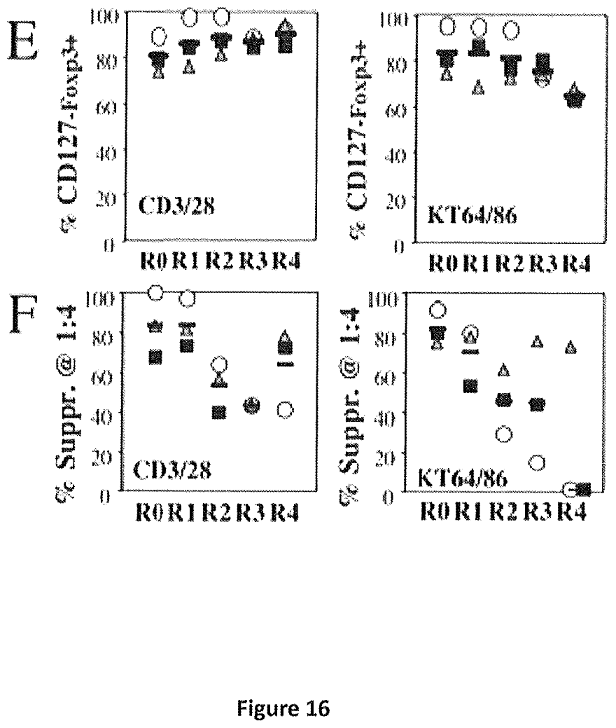

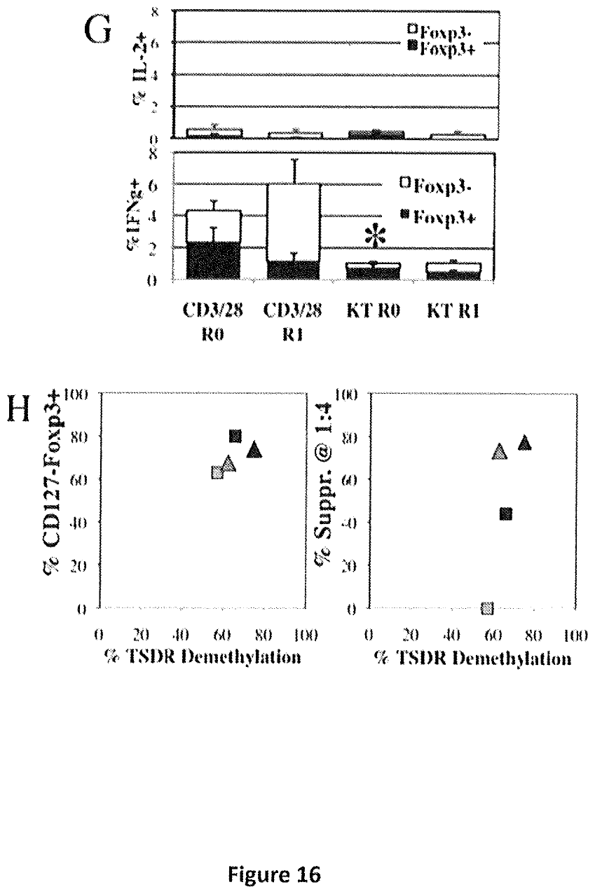

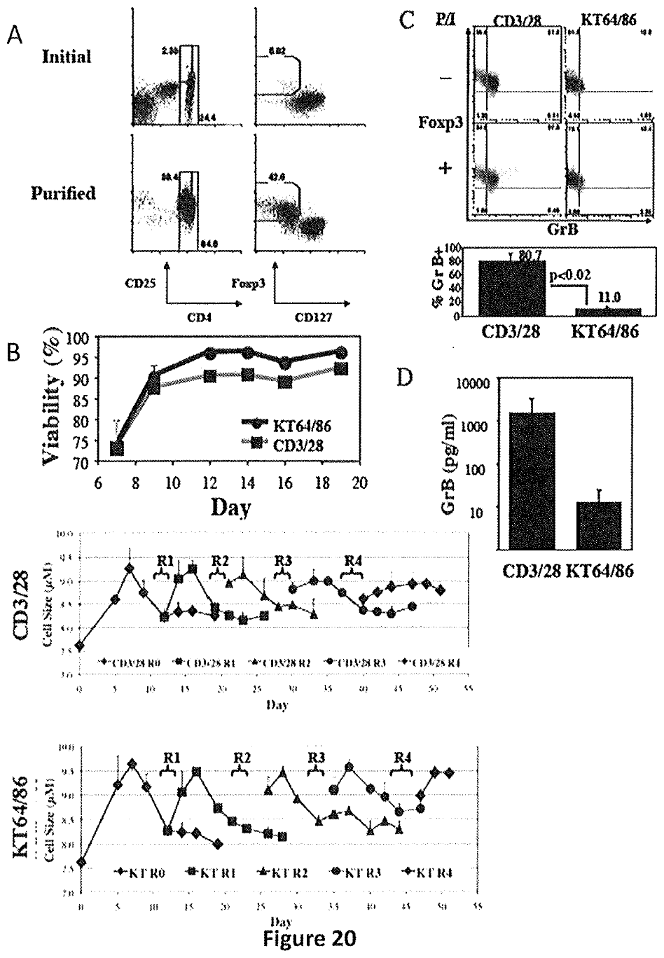

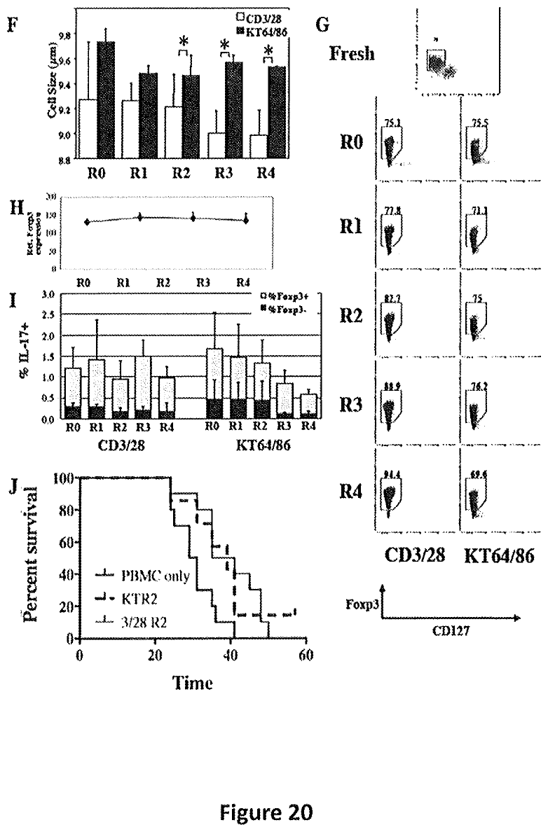

To determine whether nTreg yield could be increased if the source for the cells was changed from UCB to PB, cells were purified from leukapheresis products using a two-step protocol using GMP antibody-coated magnetic beads, whereby CD4⁺ cells were enriched by depleting cells expressing CD8, CD14, and CD19, followed by selection of CD25⁺⁺ cells (FIG. 16, (A)). Starting purity of PB nTregs was assessed by flow cytometry using a phenotype that displays potent suppressive capacity (CD4⁺127⁻ Foxp3⁺, FIG. 20, (A)) (Liu et al., 2006, J Exp Med 203(7): 1701-11), and was comparable to previous observations for UCB nTregs (95±1% CD4+, of which 66±2% were CD127⁻Foxp3⁺). Of the non-CD4+ cells in either cellular preparation, <1% were positive for CD8, −14, −19, or −56 (not shown). The average yield of PB nTregs after expansion (233+31×10⁶ cells) was ˜40-fold higher than with UCB nTregs (Brunstein et al., 2011, Blood 117(3): 1061-70).

Purified cells were stimulated with clinical-grade anti-CD3/28 mAb coated beads or KT64/86 cells, which is a cell-based artificial antigen presenting cell (aAPC) expressing CD86 (a CD28 ligand) and CD64 (the high affinity Fc receptor) via lentiviral gene transfer. KT64/86 cells were loaded with anti-CD3 mAb. IL-2 (300U/ml) and Rapamycin (109 nM) were added to all cultures (FIG. 16, (B)). As reported (Golovina, et al., 2008, Journal of Immunology 181(4):2855-68), stimulation with KT64/86 cells versus anti-CD3/28 mAb coated beads increased PB nTreg expansion by ˜5-fold (82±11 vs. 18+5-fold, respectively) (FIG. 16, (C) and (D)). The more robust expansion observed with KT64/86 cells versus anti-CD3/28 mAb coated beads was associated with increased overall viability (94.3±0.5% vs. 90.0±0.3%, p≤0.001) and decreased Granzyme B (FIG. 20, (B)-(D)). nTreg cultures stimulated once with anti-CD3/28 mAb-coated beads or KT64/86 maintained nTreg phenotype (97±2 or 99.5±0.3% CD4⁺ of which 81±5% or 84±7% were CD127⁻Foxp3⁺, respectively) and in vitro function (84+12% or 83=6% inhibition of CFSE-labeled CD8 T cell proliferation after anti-CD3 stimulation at a 1:4 ratio of nTreg:PB mononuclear cells, PBMNC) (FIG. 20, (E) and (F)). These data demonstrate that suppressive, Foxp3⁺ nTregs can be expanded from PB using GMP procedures, and shows that stimulation with cell-based aAPC is optimal. However, because PB nTreg expanded 5-10 fold less than UCB nTregs expanded without Rapamycin, overall nTreg yield was not substantially increased.

Re-Stimulation Greatly Increases nTreg Expansion

To attempt to maximize yield, experiments were performed to re-stimulate GMP bead-purified nTreg grown in Rapamycin after they had returned to resting size (≤8.5 μm FIG. 20, (E)), which has been shown to maximize CD4⁺ T cell expansion (Levine et al., 1997, J Immunol 159(12):5921-30). nTreg stimulated with KT64/86 cells were found to have a higher peak cell size as compared to anti-CD3/28 mAb coated beads (FIG. 20, (F)). Re-stimulation with anti-CD3/28 mAb coated beads or KT64/86 cells increased expansion 18- and 36-fold, respectively, to a total of 330- or 3,000-fold over input cell number (FIG. 16, (C) and (D)). Cultures remained >65% FoxP3+ and suppressed in vitro T cell proliferation >50% at ratios of 1:4 (nTreg:PBMNC) (FIG. 16, (E) and (F)). Of the non-CD4+ cells expanded with either stimulus, <1% were positive for CD8, CD14, CD19, or CD56.

Others have shown that nTreg re-stimulation in the absence of Rapamycin results in up to 30% cells that secrete either IL-2 and/or IFNγ, two cytokines that could potentially exacerbate GVHD (Hoffmann et al., 2009, Eur J Immunol 39(4):1088-97; Tran et al., 2009, Blood 113(21):5125-33). Therefore, experiments were conducted to quantitate the number of IL-2 and IFNγ secreting cells by intracellular cytokine staining after PMA/Ionomycin stimulation of bead-purified nTreg cultured with anti-CD3/28 mAb coated beads or KT64/86 cells and Rapamycin. As shown in FIG. 16, (G), <1% of cells expanded with either anti-CD3/28 mAb coated beads or KT64/86 cells secreted IL-2. In addition, while less than 6% of cells in any culture expanded with either aAPC or Rapamycin were IFNγ+, significantly fewer IFNγ+ cells were found in nTreg cultures expanded with KT64/86 than CD3/28 beads (1% vs. 4% and 1% vs. 6%±re-stimulation, respectively).

### Multiple Re-Stimulations Lead to Reduced Suppressive Function Despite Significant Presence of Foxp3

To determine whether bead-purified nTreg could be expanded even further, experiments were conducted to stimulate the above cultures another three times (4 re-stimulations total) (FIG. 16, (C)). In contrast to anti-CD3/28 mAb coated bead expanded cultures, whose peak size declined after each stimulation and was <9.0 μm after the fourth re-stimulation, peak size after KT64/86 cell stimulation remained high at ˜9.5 μm (FIG. 20, (E) and (F)). However, the fold expansion induced by successive stimulations with anti-CD3/28 mAb coated beads decreased more rapidly than with KT64/86 cells, ultimately resulting in 200-fold lower total expansion than with KT64/96 cells (25,000-fold vs. ˜ 5 million-fold, respectively) (FIG. 16, (C) and (D)). nTregs re-stimulated with anti-CD3/28 mAb coated beads remained >80% Foxp3+, and while expression gradually decreased in KT64/86 cell expanded nTregs, FoxP3 remained >60% after the fourth re-stimulation (FIG. 16E). nTregs expanded with anti-CD3/28 mAb coated beads also had higher Foxp3 levels on a per cell basis than those expanded with KT64/86 cells (FIG. 20, (G) and (H)). Despite achieving Foxp3 purities previously associated with significant suppressive function by expanded UCB nTregs (Brunstein et al., 2011, Blood 117(3):1061-70), <50% suppression was observed in 2/2 and 1/3 cultures re-stimulated 3 or 4 times with CD3/28 beads, respectively; and 2/3 and 2/3 with KT64/86 cells (FIG. 16, (F)).

Stable expression of Foxp3 is a trait of natural, but not induced, Tregs and is conferred through epigenetic modification of the Foxp3 gene at the Treg-specific demethylated region (TSDR) (Huehn et al., 2009, Nat Rev Immunol 9(2):83-9). To assess the methylation status of the Foxp3 gene in re-stimulated nTreg, DNA from cultures receiving 3 or 4 re-stimulations was purified, bisulfite modified, sequenced, and the average % methylation of 11 CpG sites contained in the Treg-specific demethylated region (TSDR) was determined. Because Foxp3 is on the X chromosome and becomes hyper-methylated during X-inactivation, the data shown are restricted to male samples. Although only two samples were evaluable, FIG. 16, (H) suggests that TSDR demethylation status is proportional to Foxp3 levels and slightly decreases between the third and fourth re-stimulation (r=0.65, P=0.35). As observed for Foxp3, TSDR demethylation is not directly proportional to suppressive function (r=0.75; P=0.25).

Sort-Purified nTreg Maintain Foxp3 Levels and Suppressive Function after Multiple Stimulations

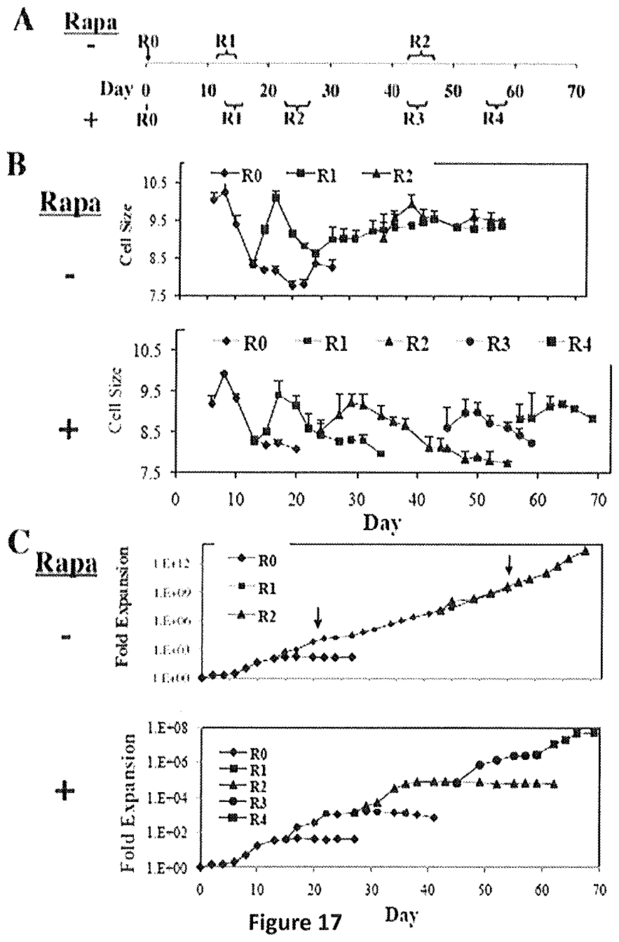

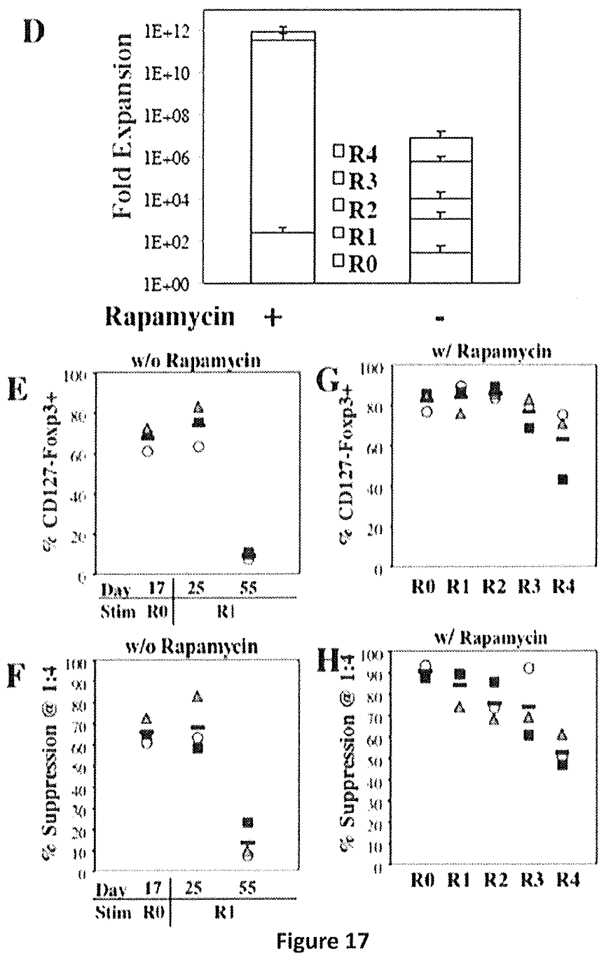

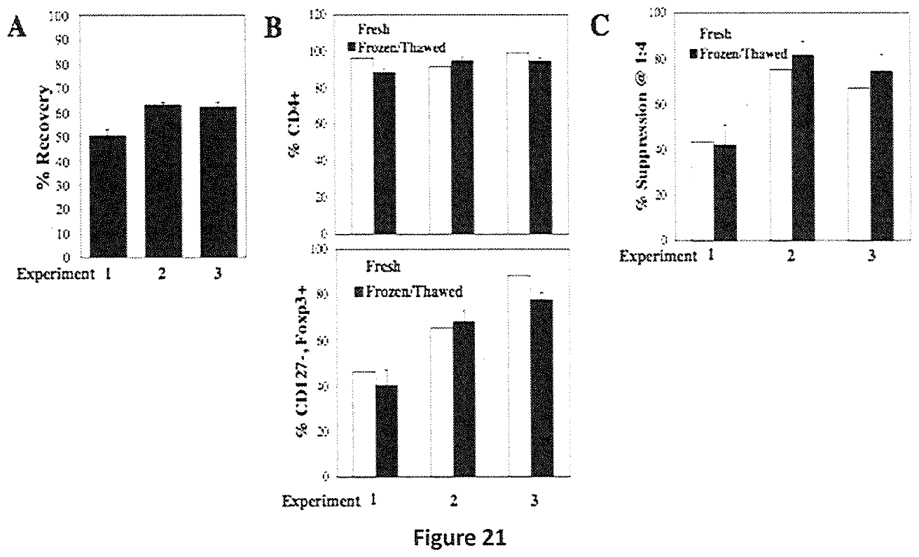

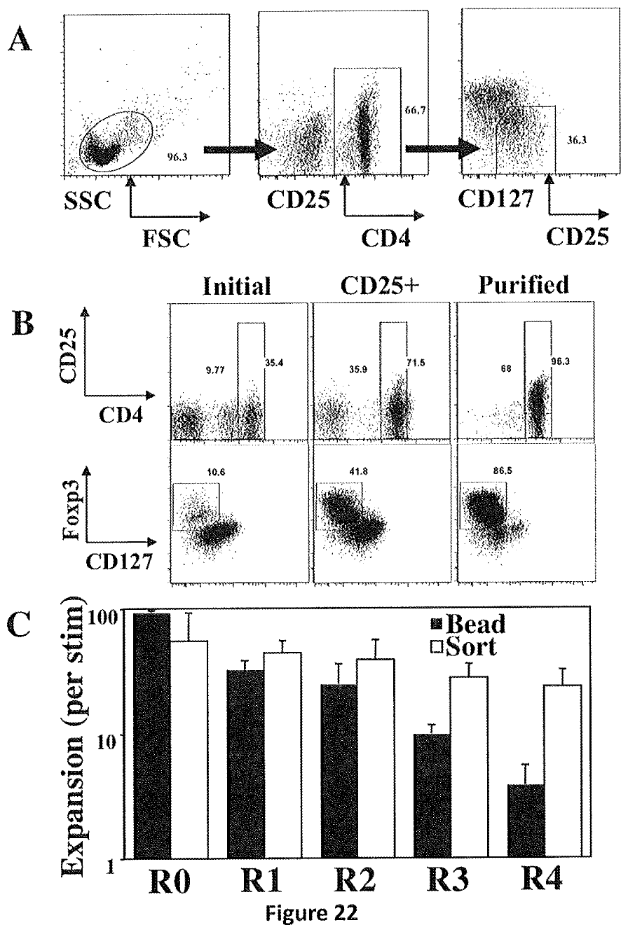

Without wishing to be bound by any particular theory, it is believed that decreased suppressive function could be caused by contaminating cells that become amplified after re-stimulation and acquire FoxP3 during the process of massive cell expansion. Therefore, PB nTregs were purified by flow cytometry sorting and re-stimulated with KT64/86 cells in the presence or absence of Rapamycin (FIG. 17, (A)). To enable more meaningful comparisons of the various re-stimulations, experiments were designed to develop freeze/thaw conditions that allowed nTregs to maintain phenotype and suppressive function so that all samples are assayed simultaneously (FIG. 21). The most common strategy for sorting nTreg is to first purify CD4+ cells, and then target on the 2% of cells with the highest expression of CD25. While cells purified in this manner are regularly >90% Foxp3+, it is relatively inefficient and only captures approximately 20% of the total Foxp3+ cells. To maximize yield, an initial purification with magnetic anti-CD25 beads and then sorted for CD4+25++127-cells was performed, which allowed >25% of sorted cells to be positively selected (vs. 2% for CD25++ sorting). This method increased initial nTreg purity from 66+2% CD127 Foxp3 for bead-based purification to 84±3 (p<0.003), and resulted in a routine yield of 15-30×10⁶ nTregs from 2×10⁹ PBMNC (FIG. 22, (A) and (B)).

nTregs stimulated with KT64/86 cells in the absence of Rapamycin, which is known to affect size and proliferation (Rathmell et al., 2001, J Immunol 167(12):6869-76), had both a larger peak size (FIG. 17, (B); 10.4 vs. 9.9 μM for without and with Rapamycin, respectively; p<0.01), and increased expansion (FIGS. 17, (C) and (D); 290-vs. 55-fold, respectively). nTregs cultured in the presence or absence of Rapamycin were re-stimulated at 8.5 μm (day 13±1) and, after 4 days of blasting, cultures started to decrease in size and stopped expanding. However, after day 25, without additional stimulation, cultures grown without Rapamycin increased in size to about 9.3 μM and started proliferating, impressively expanding over 5×10¹¹-fold throughout the 55 day observation period. Additional re-stimulation did not increase either cell size or maximal expansion. Day 55 cultures contained few CD127−Foxp3+ nTreg cells and were not suppressive, whereas those harvested on day 25 had still expanded 11,000±2,000-fold, were ≥60% CD127−Foxp3+ and conferred ≥60% suppression of T cell proliferation at 1:4 (nTreg:PBMNC) (FIGS. 17, (E) and (F)).

In contrast to cultures established in the absence of Rapamycin, sort-purified nTreg expanded with KT64/86 cells in the presence of Rapamycin returned to resting size and ceased proliferating after each re-stimulation (FIGS. 17, (B) and (C)). Cumulative expansion after re-stimulation of sort-purified nTregs+ Rapamycin was >6-fold higher than bead-purified nTregs (31±14×10⁶ vs. 4.7±0.7×10⁶ fold expansion, respectively, p<0.05), due mainly to the fact that the fold-expansion did not decline after each re-stimulation (FIG. 22, (C)). Repeated stimulation caused a gradual decrease in Foxp3 levels such that after the fourth re-stimulation, only 63±12% of cells were CD127⁻ Foxp3⁺ (FIG. 17, (G)). However, unlike bead-purified cultures performed under the same conditions, sort-purified nTregs expanded after four repetitive stimulations maintained >50% suppression of T-cell responses at 1:4 for all re-stimulations (FIG. 17, (H)).

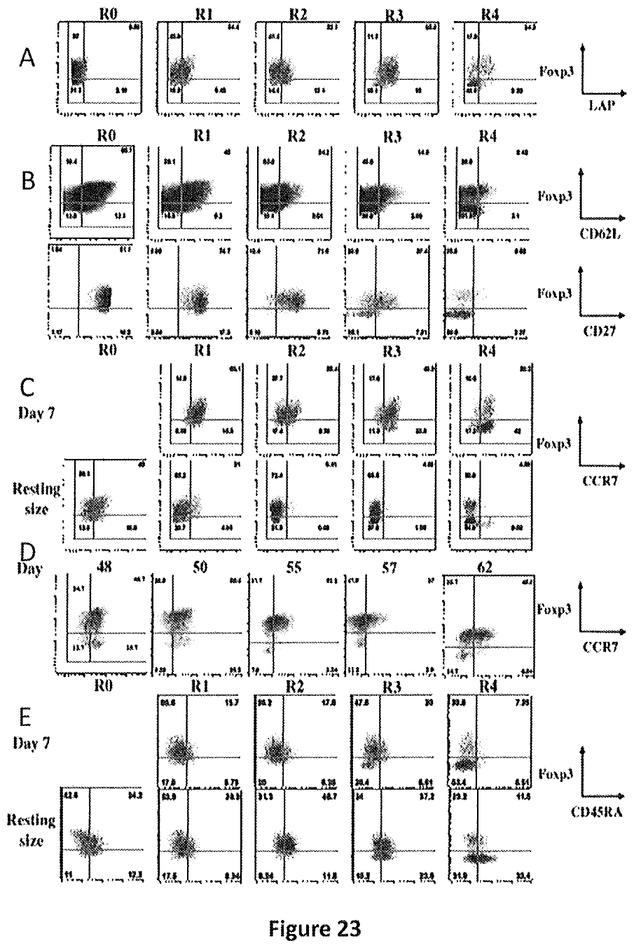

To determine whether re-stimulation affects the Treg phenotype, the level of several Treg associated markers (including LAP, CD62L, CD27, CCR7 and CD45RA) was assessed on cells receiving a single vs. multiple stimulations. While Latency Associated Peptide (LAP), derived from the N-terminal region of TGFβ, was expressed on Foxp3+ cells after all 4 re-stimulations (FIG. 23, (A)), CD62L and CD27 staining was lost after 2 and 4 re-stimulations, respectively (FIG. 23, (B)). CCR7 behaved like an activation marker, being more highly expressed at day 7 compared to that in resting size (FIG. 23, (C)). However, if nTregs were maintained in culture after returning to basal size, a subpopulation of nTregs spontaneously regained CCR7 staining (FIG. 23, (D)). While it is not surprising that re-stimulation decreased levels of CD45RA (expressed on naïve, resting T cells and Tregs), the finding that cells regained staining after returning to resting size was unanticipated and not obvious (FIG. 23, (E), especially re-stimulations 2 and 3).

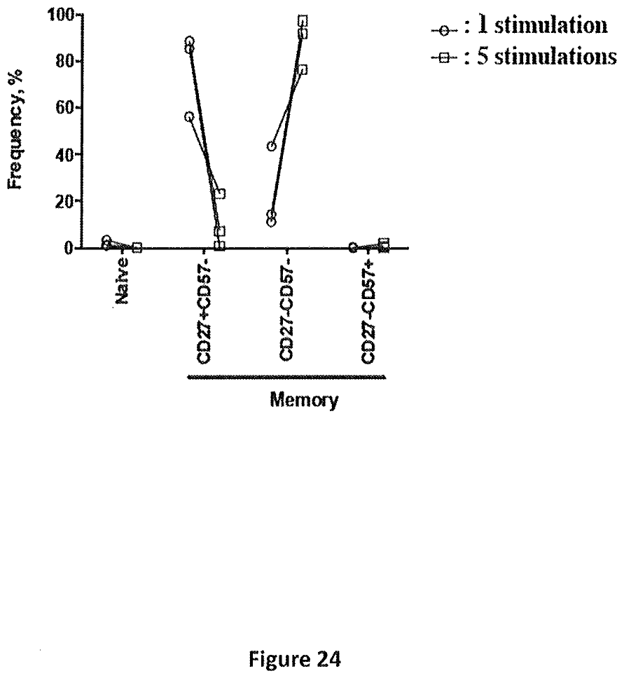

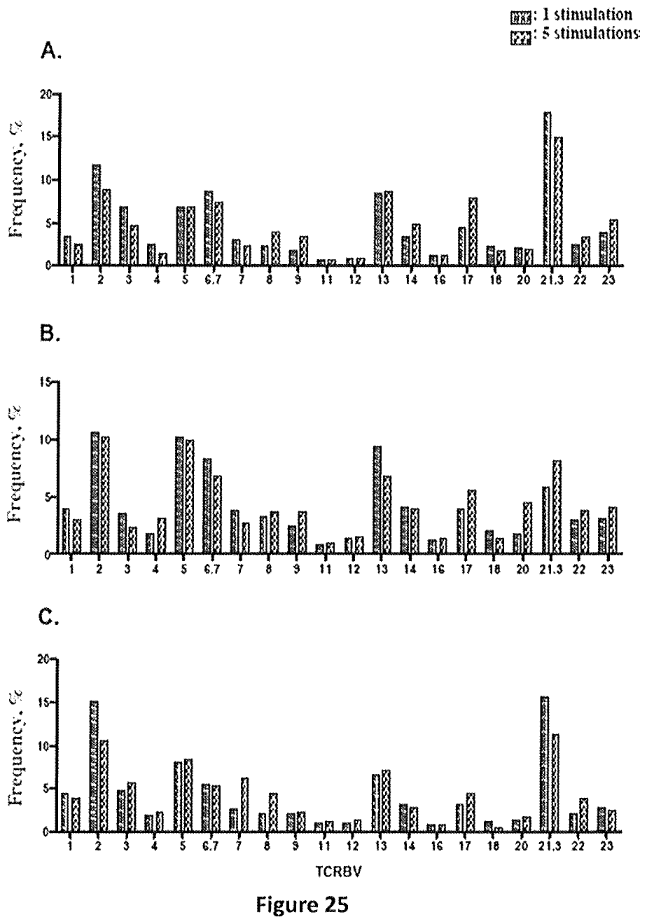

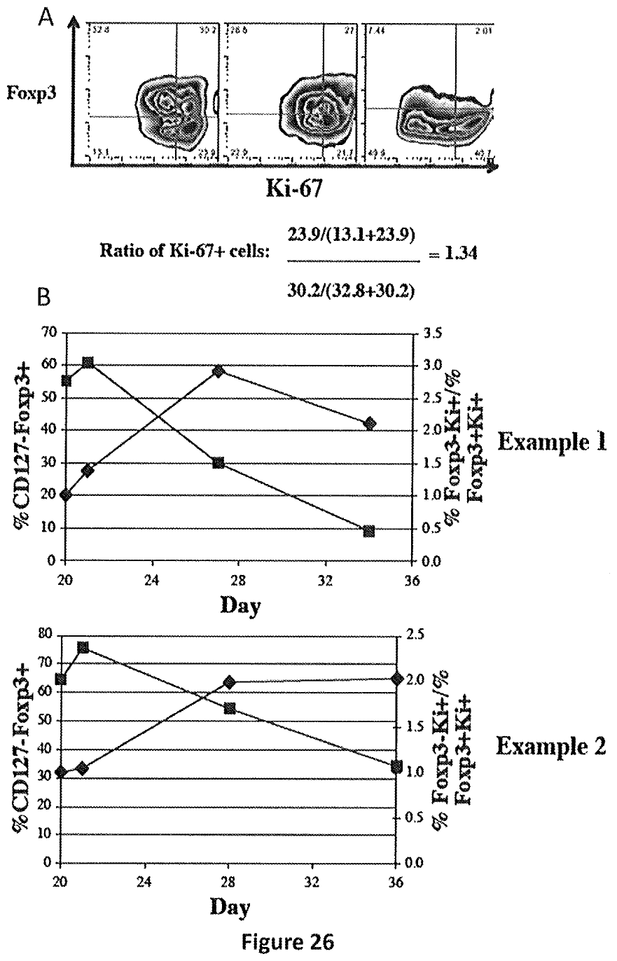

The next set of experiments was performed to examine changes in surface phenotype and T-cell receptor (TCR) repertoire of the nTregs expanded with one (R0) or a total of 5 stimulations (R4). After multiple rounds of stimulation, the nTreg changed from CD27+CD45RA-CD57-to CD27-CD45RA-CD57-, suggesting that they were undergoing differentiation to a more mature state (FIG. 24). However, an increase in CD57 expression was not noted after expansion, suggesting that the cells did not become terminally differentiated or senescent (Brenchley et al, 2003, Blood 101(7):2711-20). Finally, TCR VB usage before and after expansion was essentially unchanged suggesting that particular TCR VB families were not preferentially expanded (FIG. 25).

nTreg Cultures Re-Stimulated with KT64/86 Cells in the Presence of Rapamycin do not Secrete IL-2 or Effector Cytokines

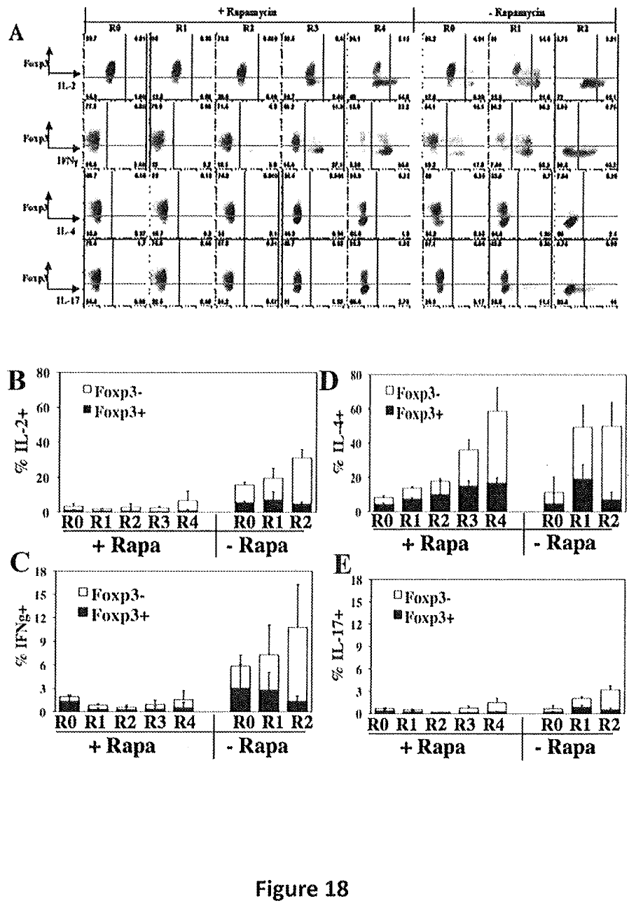

Repetitive stimulation of TH cells in the absence of Rapamycin generates effector cells, which secrete cytokines that could exacerbate GVHD. To determine the extent of effector T-cell contamination in the cultures, samples of each re-stimulation from KT64/86 expanded cultures grown with or without Rapamycin were stimulated with PMA/Ionomycin and assayed for IL-2, IL-4, IL-17 and IFNγ (FIG. 18, (A)) using intracellular cytokine staining. To make comparisons between various re-stimulations more valid, frozen nTreg representing all conditions were assayed simultaneously, and were co-stained for Foxp3 to differentiate secretion by nTregs vs. contaminating cells. The addition of Rapamycin suppressed effector cell generation such that≤3% of PMA/ionomycin stimulated cells secreted IL-2 and ≤2% secreting IFNγ, as compared to ≥17% and ≥6% for cultures without Rapamycin (FIGS. 18, (B) and (C)). In contrast, Rapamycin was less effective at inhibiting IL-4 production in Foxp3⁺ or Foxp3⁻ cells, and the percentage of IL-4+ cells increased with each successive re-stimulation from 8±2% to 58±17% (FIG. 18, (D)). The total number of IL-17 secreting cells present in cultures of sorted nTreg was consistently low (<3.1%) for all stimulations with or without Rapamycin (FIGS. 18, (E)).

nTregs Expanded with Multiple Rounds of Stimulation Ameliorate Disease in a Xenogeneic Model of GVHD

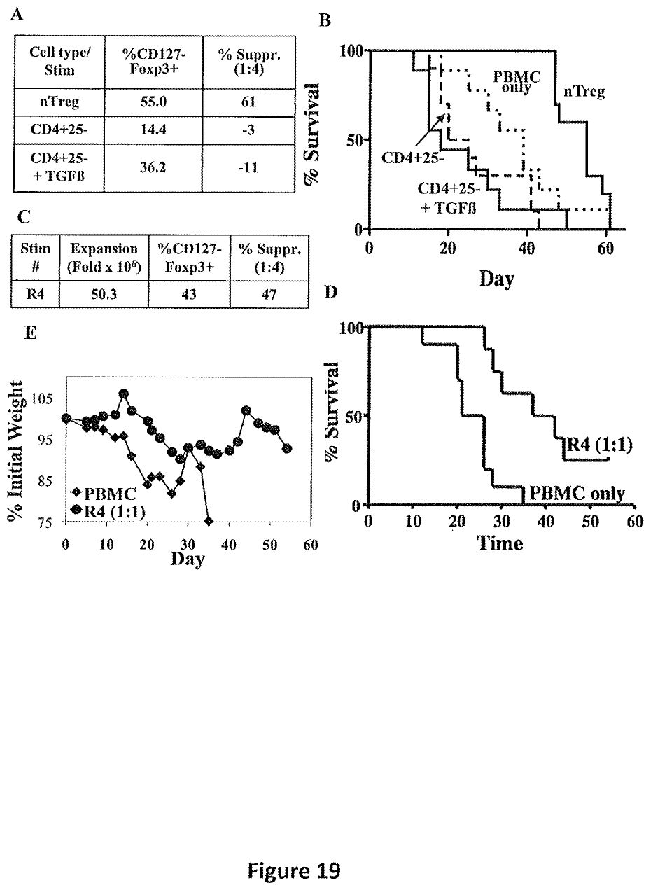

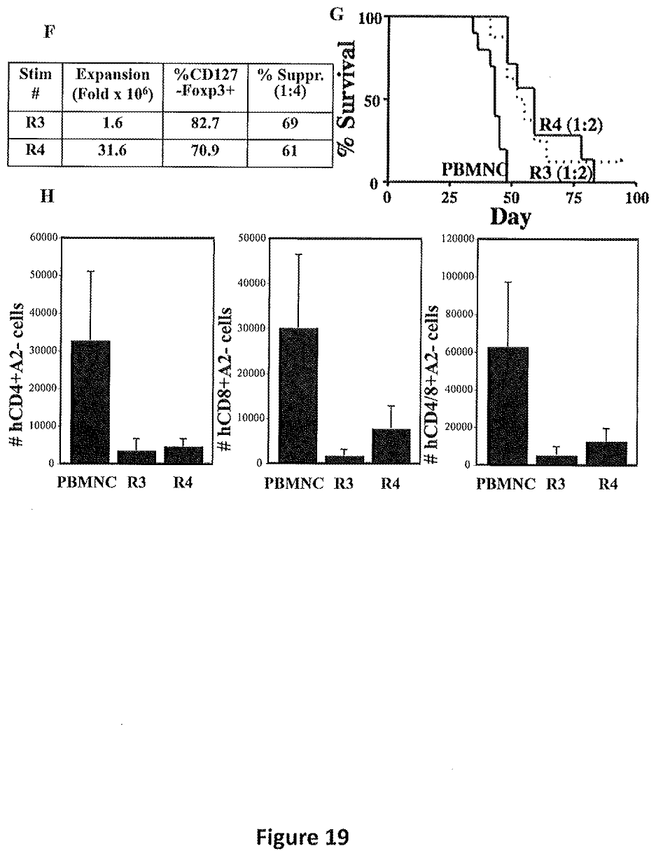

Several groups have reported that nTreg are not terminally differentiated and can be reprogrammed into Teff cells (Beriou et al., 2009, Blood 113(18):4240-9; Koenen et al., 2008, Blood 112(6):2340-52; Radhakrishnan et al., 2008, J Immunol 181(5):3137-47), which are capable of inducing disease (Radhakrishnan et al., 2008, J Immunol 181(5):3137-47). Therefore, a xenogeneic model of GVHD was used, in which nTreg are co-transferred at a 1:1 ratio with allogeneic PBMNCs (30×10⁶ each) into NOD/Scid/γ_{c}^{-/-} recipients, to compare the stability and safety of in vitro expanded nTregs versus CD4+25− cells that were re-stimulated 4 times cultured in the absence or presence of TGFβ, the latter used to induce FoxP3 (FIG. 19, (A)). The adoptive transfer of nTregs increases median survival from 39 to 55 days (p<0.01). Transfer of non-Tregs appears to exacerbate GVHD, even if Foxp3 is induced with TGFβ (FIG. 19, (B)), whereas Foxp3− cells present in nTreg cultures did not expand or persist long-term and, in contrast to cultures expanded from CD4+25− cells, did not exacerbate GVHD. The in vivo potency, stability and safety of nTreg expanded 50-million fold with four re-stimulations using KT64/86 cells was also tested. While recipients of PBMNC only rapidly and uniformly succumbed to GVHD, mice given nTregs had a significantly prolonged survival and 25% of mice survived long-term (FIG. 23, (D); n=8-10/group; p<0.05). GVHD amelioration was also indicated by a significant decrease in weight loss between days 14 and 21 (FIG. 23, (E)).

The partial protection observed using nTregs in this xenogeneic GVHD model has also been observed using UCB nTregs obtained after a single stimulation with anti-CD3/28 mAb coated beads, which we have shown to rescue 50% of macrophage-depleted, sublethally irradiated Rag2, γ_{c}^{-/-} recipients when infused at a 1:1 ratio with PBMNC (Hippen et al., 2008, Blood 112(7):2847-57).

Since there was a modest decrement in CD127⁻ FoxP3⁺ and in vitro suppression in nTreg GVHD model, a suboptimal ratio of nTreg:PBMNC (1:2) was used to help stratify potential differences. FIG. 19, (F) shows that the characteristics of nTregs re-stimulated three or four times maintained their phenotype and in vitro suppressive function after cryopreservation and thawing. Both expanded nTreg preparations significantly reduced GVHD-induced lethality versus PBMNC only controls and there was no difference in their relative potency (FIGS. 19, (G); P<0.003 and 0.001 vs. PBMNC controls for three or four re-stimulations, respectively) or prevention of weight loss (data not shown). Expansion of PB-derived CD4 and CD8⁺ T-cells is predictive of GVHD severity and FIG. 19, (H) shows that, like UCB nTreg, co-transfer of re-stimulated PB nTregs significantly reduced the number of GVHD-causing T-cells on day 30 post-transfer.

Therapeutic Use of nTregs

The therapeutic potential of nTregs to prevent or cure multiple autoimmune diseases or GVHD in murine or xenogeneic models has been well documented (Hoffmann et al., 2002, J Exp Med 196(3):389-99; Shevach et al., 2006, Immunol Rev 212:60-73; Taylor et al., 2002, Blood 99:3493-9). Two critical obstacles that need to be overcome before implementing this therapy for humans are generating sufficient cell numbers and demonstrating their in vivo safety and stability. The results presented herein demonstrate that sort-purified nTregs can be expanded at least 50 million fold by repetitive stimulation with a cell-based aAPC while maintaining suppressive function in vitro and in vivo.

The results presented herein also demonstrated that Rapamycin minimized contamination with Th1 inflammatory cytokine secreting cells, but not Th2, which has anti-inflammatory properties. Re-stimulated nTreg differentiated from CD27+ memory to CD27− memory but, importantly, did not adopt a senescent (CD57+) phenotype (Klebanoff et al., 2006, Immunol Rev 211:214-24). The lack of VB skewing in the T cell receptor repertoire suggests that the expanded nTreg retained a broad spectrum of reactivities, but were not transformed. While GMP sorting can be challenging for many institutions, the massive expansion driven by re-stimulation according to the methods discussed herein allows the creation of a master cell bank in a central facility that has a GMP flow cytometer and used to treat high numbers of patients.

Maximizing nTreg expansion while minimizing loss of suppressive function and contamination with non-Tregs is critical for using expanded nTregs for cellular therapy. Three studies have shown that nTregs can be expanded >1000-fold if re-stimulated in the absence of Rapamycin, but in each case, the cultures contained significant numbers of IL-2 and IFNγ secreting cells that were both Foxp3− and +(Hoffmann et al., 2009, Eur J Immunol 39(4): 1088-97; Tran et al., 2009, Blood 113(21):5125-33; Putnam et al., 2009, Diabetes 58(3):652-62). The results presented herein confirmed these data and found that nTreg cultures eventually lost Foxp3 and suppressive function in the absence of Rapamycin. Loss of Foxp3 correlated with an increased ratio of cycling (i.e. Ki-67+) Foxp3− cells (FIG. 25), suggesting loss of purity is due to outgrowth of Foxp3− cells as opposed to conversion of Foxp3+ cells as suggested by one report (Hoffmann et al., 2009, Eur J Immunol 39(4): 1088-97).

It has been demonstrated that the increased stimulatory capacity of cell-based aAPC allowed PB nTregs to be expanded 1000-fold with a single re-stimulation, even in the presence of Rapamycin, and nTreg expanded with aAPC were equal to CD3/28 expanded cells at suppressing xenogeneic GVHD (FIG. 20J and (Golovina, et al., 2008, Journal of Immunology 181(4):2855-68)). For these initial studies, re-stimulation was performed at the growth plateau phase, but the high variability (day 8 to 12) and ambiguity in identifying a plateau are not conducive to clinical production. Re-stimulating on a specific day is optimal for clinical trials, but while studies without Rapamycin showed re-stimulation on day 7 increased expansion, no increase in expansion was seen with this single re-stimulation at this fixed time point (average total expansion of 25-fold for bead-purified nTreg expanded with CD3/28 beads; n=3). Although re-stimulation based upon cell size resulted in more variability in the day of re-stimulation than would be the case at a single time point, such an approach identified a time range (day 13±1) more suitable for clinical re-stimulation.

All nTreg cultures contained Foxp3− cells, which have the potential, especially after re-stimulation, to become effector T cells and exacerbate disease. While nTreg cultures re-stimulated in the absence of Rapamycin contained significant numbers of IL-2 and IFNγ secreting cells, these cells were almost nonexistent in the presence of Rapamycin. Furthermore, when transferred in vivo, Foxp3− cells present in nTreg cultures did not expand or persist long-term and, in contrast to cultures expanded from CD4+25− cells, did not exacerbate GVHD. In addition, studies show Rapamycin temporally imparts Foxp3 expression and Treg-like activity to effector cells which can revert to effector cells if Rapamycin is removed (Valmori et al., 2006, J Immunol 177(2):944-9). LAP expression differentiates activated natural Treg from stimulated CD4+25− T cells expressing Foxp3 spontaneously or after exposure to TGFβ or Rapamycin (Tran et al., 2009, Blood 113(21):5125-33). Nearly all Foxp3+ cells expressed LAP even 7 days after re-stimulation, showing that the cultures remain natural Treg. Furthermore, cultures expanded over 1 million-fold maintained nTreg specific demethylation in the Foxp3 gene. Murine T cells expanded in Rapamycin are Th2 skewed, secrete IL-4 and IL-10 and, after adoptive transfer, decrease allospecific IFNγ secretion and ameliorate disease in a murine model of GVHD (Foley et al., 2008, Biol Blood Marrow Transplant 14(9):959-72). Interestingly while Rapamycin almost completely inhibited the differentiation of IL-2 and IFNγ secreting cells, the effect on IL-4 was not complete, and >50% of cells secreted IL-4 (Foxp3+ and -) after the fourth re-stimulation.

Murine and human Tregs are not terminally differentiated, and can be reprogrammed to secrete IL-17 in vitro or in vivo when activated in the presence of IL-6 (Koenen et al., 2008, Blood 112(6):2340-52; Radhakrishnan et al., 2008, J Immunol 181(5):3137-47; Beriou, et al., 2009, Blood 113(18):4240-9). Adoptive transfer of reprogrammed murine Tregs induced autoimmune diabetes but, unlike their human counterparts, these cells also produced IFNγ and TNFα. It is not known whether reprogrammed human Tregs will cause disease, since only ˜5% of nTregs become IL-17+ in vitro (Koenen et al., 2008, Blood 112(6):2340-52) and these retain suppressive function (Beriou, et al., 2009, Blood 113(18):4240-9). Several findings from this study suggest nTreg reprogramming may not be a grave issue in developing a cellular therapy for in vitro expanded nTregs. First, IL-17 was undetectable in the supernatants of all re-stimulation samples cultured with Rapamycin. Second, the number of expanded cells that were IL-17+ cells was very low (<2% total and ≤0.5% Foxp3+IL-17+) and, even more important, did not increase significantly over the 4 re-stimulation cycles. Although the likelihood for in vivo reprogramming of nTregs and especially expanded nTregs may be context dependent, the high degree of TSDR demethylation of these cells may provide some degree of resistance to the reprogramming process.

In summary, this degree of expansion will revolutionize nTreg cellular therapy for GVHD and graft rejection by allowing the creation of an off the shelf therapy using nTreg banks generated from HLA typed donors with known safety and potency records. The massive expansion observed with repetitive polyclonal stimulation should also allow relatively rare, auto-antigen specific nTreg clones to be expanded to treat autoimmune diseases. Ultimately, this strategy can be applied to expansion of antigen specific nTreg that are more effective than polyclonal Tregs at suppressing disease and potentially preferable to induced Tregs induced in vitro by FoxP3 gene transfer or other conditions that favor FoxP3 expression. If increased purity and/or suppressive function is required, nTregs could be re-isolated after expansion using a protocol described recently by Shevach's group based upon LAP expression (Tran et al., 2009, Blood 113(21):5125− 33).

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1-23. (canceled)

24. A method for expanding natural regulatory T cells (nTregs), the method comprising:
stimulating an initial cell population comprising nTregs in a culture with a first agent that provides a primary activation signal, a second agent that provides a co-stimulatory signal, and rapamycin to generate a population of cells comprising proliferated nTregs; and
re-stimulating the population of cells comprising proliferated nTregs with the first agent, the second agent, and the rapamycin to generate an expanded population of cells comprising nTregs expressing FoxP3 and exhibiting suppressor activity, wherein nTreg cell number in the expanded population of cells is increased by about 100-fold or more as compared with the nTreg cell number present in the initial cell population.

25. The method of claim 1, wherein the re-stimulating is performed when proliferation of the population of cells has decreased based upon a desired cell size.

26. The method of claim 25, wherein the desired cell size comprises a cell size associated with a resting nTreg.

27. The method of claim 26, wherein the cell size comprises about 8.5 μM.

28. The method of claim 24, wherein the initial cell population is derived from umbilical cord blood cells.

29. The method of claim 24, wherein the initial cell population was previously cryopreserved.

30. The method of claim 24, wherein the method is repeated at least four times.

31. The method of claim 24, wherein the first agent comprises an anti-CD3 antibody and wherein the second agent comprises a molecule that binds with CD28.

32. The method of claim 31, wherein the first agent and the second agent are co-immobilized on a first surface.

33. The method of claim 31, wherein the molecule that binds CD28 is selected from the group consisting of anti-CD28 antibody, B7 (CD80), B7-2 (CD86), and any combination thereof.

34. The method of claim 24, wherein the rapamycin is used at a concentration of 109 nanomolar (nM).

35. The method of claim 24, wherein the expanded population of cells substantially retains a nTreg phenotype.

36. The method of claim 24, wherein the expanded population of cells does not secrete IFNγ and IL-2.

37. The method of claim 24, wherein the expanded population of cells has not substantially reverted to T effector phenotype.

38. The method of claim 24, further comprising cryopreserving the expanded population of cell.

39. The method of claim 38, further comprising thawing and administering the expanded population of cells to a subject in need thereof.
